(19) United States
(12) Patent Application Publication
(10) Pub. No.: US 2025/0066800 A1
(43) Pub. Date: Feb. 27, 2025

(54) Functional DNA Cassette and Plasmid

(71) Applicant: Moderna Enzymatics Co., Ltd., Tokyo (JP)

(72) Inventors: Masayuki Suetsugu, Tokyo (JP); Takahito Mukai, Tokyo (JP)

(21) Appl. No.: 18/726,215

(22) PCT Filed: Jan. 19, 2023
(86) PCT No.: PCT/JP2023/001521

§ 371 Date: Jul. 2, 2024

(30) Foreign Application Priority Data
Jan. 19, 2022 (JP) 2022-006523

(51) Int. Cl. C12N 15/70; C12P 19/34

(52) U.S. Cl. CPC C12N 15/70; C12P 19/34

(57) ABSTRACT

The present invention mainly relates to a DNA cassette comprising: a replication origin sequence capable of binding to an enzyme with DnaA activity; and a first promoter sequence; wherein transcription from the first promoter sequence flows into the replication origin sequence, and the distance between the 3′ terminal base of the first promoter sequence and the terminal base of the replication origin sequence is within 450 bases. The invention also relates to a plasmid comprising: a replication origin sequence capable of binding to an enzyme with DnaA activity; a first promoter sequence; and a plasmid replication origin; wherein transcription from the first promoter sequence flows into the replication origin sequence, and the distance between the 3′ terminal base of the first promoter sequence and the terminal base of the replication origin sequence is within 2000 bases.

### TECHNICAL FIELD

The present invention primarily relates to a DNA cassette comprising a replication origin sequence capable of replicating within bacteria and a promoter sequence, as well as a plasmid.

### BACKGROUND ART

DNA cloning technology, which forms the foundation of biotechnology development, is a method of amplifying circular DNA prepared by cutting and pasting DNA fragments as plasmids within cells such as Escherichia coli. Recently, plasmid DNA has been used not only in genetic engineering research but also as a raw material for gene therapy. Therefore, there is a need for technology that allows the high-purity and large-scale preparation of plasmid DNA from host cells such as Escherichia coli.

One of the key points in high-purity large-scale preparation is to increase the copy number of plasmids maintained per host cell. Some plasmids, such as those derived from the Escherichia coli F factor, are maintained at one or two copies per host cell, while others, such as the colicin E1 factor (ColE1) plasmid, are maintained at several dozen to several hundred copies per host cell. For example, pUC and pBR322, commonly used for large-scale preparation of plasmids, are ColE1-type plasmids (Non-Patent Literatures 1 and 2).

As a method for amplifying circular DNA in vitro, the replication cycle reaction (RCR) amplification method is known (Patent Literatures 1 to 3). The RCR amplification method is a technique that replicates circular DNA comprising oriC, which can bind to an enzyme with DnaA activity, using an enzyme group that catalyzse the replication of circular DNA, an enzyme group that catalyzes Okazaki fragment joining reaction and synthesizes two sister circular DNAs constituting a catenane, and an enzyme group that catalyzes the separation of the two sister circular DNAs. Therefore, in the RCR amplification method, circular DNA comprising oriC is amplified. However, plasmids into which oriC is inserted are difficult to maintain in host cells at high copy numbers (Non-Patent Literature 3).

### CITATION LIST

### Patent Literature

Patent Literature 1: International Publication No. WO 2016/080424

Patent Literature 2: International Publication No. WO 2017/199991

Patent Literature 3: International Publication No. WO 2018/159669

Patent Literature 4: U.S. Pat. No. 7,575,860

Patent Literature 5: U.S. Pat. No. 7,776,532

Patent Literature 6: U.S. Pat. No. 8,968,999

Patent Literature 7: International Publication No. WO 2019/009361

Patent Literature 8: International Publication No. WO 2016/013592

### Non-Patent Literatures

Non-Patent Literature 1: Yanisch-Perron et al., Gene 1985 vol.33 p. 103-119.

Non-Patent Literature 2: Lin-Chao et al. Molecular Microbiology 1992 vol.6 p.3385-3393.

Non-Patent Literature 3: Loebner-Olesen, EMBO Journal 1999 vol. 18(6) p. 1712-1721.

Non-Patent Literature 4: de Boer et al., Proceedings of the National Academy of Sciences of the United States of America 1983 vol.80 p.21-25.

Non-Patent Literature 5: Lockshon and Morris, Journal of Molecular Biology 1985 vol. 181 p.63-74.

Non-Patent Literature 6: Folarin et al., Bioengineering 2019 vol.6(2):54.

Non-Patent Literature 7: Hiasa and Marians, The Journal of Biological Chemistry 1994 vol.269(43) p.26959-26968.

Non-Patent Literature 8: Neylon et al., Microbiology and Molecular Biology Reviews 2005 vol.69(3) p.501-526.

Non-Patent Literature 9: Vivian et al., Journal of Molecular Biology 2007 vol.370 p.481-491.

Non-Patent Literature 10: Hartley et al., Genome Research 2000 vol. 10 p. 1788-1795.

Non-Patent Literature 11: Copeland et al., Nature Reviews Genetics 2001 vol.2 p.769-779.

Non-Patent Literature 12: Su'etsugu et al., Nucleic Acids Research 2017 vol.45(20) p. 11525-11534.

### SUMMARY OF INVENTION

### Problem to be Solved by the Invention

The main objective of the present invention is to provide a plasmid with a high copy number and excellent stability in bacteria, and a DNA cassette suitable for preparing such a plasmid.

### Means for Solving the Problem

As a result of diligent research, the inventors found that a plasmid comprising: a promoter designed to direct transcription into the oriC; oriC; and a plasmid replication origin; achieves a high copy number per host cell and maintains stability within the host cell even with the presence of oriC, thus completing the present invention.

Namely, the DNA cassette and others according to the present invention are as follows in [1] to [23].
[1] A DNA cassette comprising: a replication origin sequence capable of binding to an enzyme with DnaA activity; and a first promoter sequence, wherein
   transcription from the first promoter sequence flows into the replication origin sequence, and
   the distance between the 3′ terminal base of the first promoter sequence and the terminal base of the replication origin sequence is within 450 bases.
[2] A DNA cassette comprising: a replication origin sequence capable of binding to an enzyme with DnaA activity; a first promoter sequence; and a gyrase-binding sequence, wherein
   transcription from the first promoter sequence flows into the replication origin sequence, and
   the distance between the 3′ terminal base of the first promoter sequence and the terminal base of the replication origin sequence is within 2000 bases.
[3] The DNA cassette according to [2], wherein the gyrase-binding sequence is derived from bacteriophage Mu.
[4] The DNA cassette according to any one of [1] to [3], further comprising a complementary sequence of a second promoter sequence on the 3′ side of the first promoter sequence.
[5] A plasmid comprising the DNA cassette according to any one of [1] to [4] and a plasmid replication origin.
[6] A plasmid comprising a replication origin sequence capable of binding to an enzyme with DnaA activity, a first promoter sequence, and a plasmid replication origin, wherein
   transcription from the first promoter sequence flows into the replication origin sequence, and
   the distance between the 3′ terminal base of the first promoter sequence and the terminal base of the replication origin sequence is within 2000 bases.
[7] The plasmid according to [6], further comprising a gyrase-binding sequence.
[8] The plasmid according to any one of [5] to [7], wherein the plasmid replication origin is of the ColE1 type.
[9] The plasmid according to any one of [5] to [8], wherein the distance between the first promoter sequence and the replication origin sequence is within 300 bases.
[10] A bacterium comprising the plasmid according to any one of [5] to [9].
[11] The bacterium according to [10], wherein the bacterium is Escherichia coli.
[12] A method for producing a plasmid, comprising culturing the bacterium according to [10] or [11] and recovering the plasmid from the resulting culture.
[13] A method for producing single-stranded RNA, comprising producing a plasmid by the method according to [12] and obtaining RNA by transcription from the plasmid.
[14] A method for preparing a plasmid comprising a DNA cassette, the method comprising:
   providing the DNA cassette comprising a replication origin sequence capable of binding to an enzyme with DnaA activity and a first promoter sequence, wherein transcription from the first promoter sequence flows into the replication origin sequence, and the distance between the 3′ terminal base of the first promoter sequence and the terminal base of the replication origin sequence is within 2000 bases, and
   introducing the DNA cassette into a plasmid comprising a plasmid replication origin.
[15] A method for preparing a plasmid comprising a DNA cassette, the method comprising:
   providing a DNA cassette comprising a replication origin sequence capable of binding to an enzyme with DnaA activity,
   providing a plasmid comprising: a plasmid replication origin; and a promoter sequence, and
   introducing the DNA cassette into the plasmid such that transcription from the promoter sequence of the plasmid flows into the replication origin sequence.
[16] A method for preparing a plasmid comprising a DNA cassette, the method comprising:
   providing a DNA cassette comprising: a replication origin sequence capable of binding to an enzyme with DnaA activity; and a gyrase-binding sequence,
   providing a plasmid comprising: a plasmid replication origin; and a promoter sequence; and
   introducing the DNA cassette into the plasmid such that transcription from the promoter sequence of the plasmid flows into the replication origin sequence.
[17] The method for preparing a plasmid according to [15] or [16], wherein the distance between the 3′ terminal base of the promoter sequence and the terminal base of the replication origin sequence is within 2000 bases.
[18] The method for preparing a plasmid according to any one of [15] to [17], wherein
   introducing the DNA cassette into the plasmid comprises:
   providing a reaction solution comprising the plasmid, the DNA cassette, a protein with RecA family recombinase activity, and an exonuclease, and
   incubating the reaction solution to perform homologous recombination, wherein
   the plasmid contains regions Ha and Hb, the region Hb being located downstream of the region Ha, and
   the DNA cassette contains a homologous region corresponding to the region Ha and a homologous region corresponding to the region Hb, the latter being positioned downstream of the former.
[19] A DNA cassette for preparing high-copy-number plasmid, comprising a replication origin sequence capable of binding to an enzyme with DnaA activity; and a gyrase-binding sequence, wherein
   the length of the cassette being 1000 base pairs or less.
[20] A DNA cassette comprising a replication origin sequence capable of binding to an enzyme with DnaA activity, a first promoter sequence, and a terminator sequence, wherein
   transcription from the first promoter sequence flows into the replication origin sequence,
   the distance between the 3′ terminal base of the first promoter sequence and the terminal base of the replication origin sequence is within 200 bases, and
   the terminator sequence is located downstream of the first promoter sequence, and the distance between the 3′ terminal base of the first promoter sequence and the 5′ terminal base of the terminator sequence being within 600 bases.
[21] The DNA cassette according to [20], further comprising a pair of ter sequences that are each inserted outward with respect to the replication origin sequence.
[22] A DNA cassette comprising: a replication origin sequence capable of binding to an enzyme with DnaA activity; a first promoter sequence; and a pair of ter sequences that are each inserted outward with respect to the replication origin sequence, wherein
   transcription from the first promoter sequence flows into the replication origin sequence, and
   the distance between the 3′ terminal base of the first promoter sequence and the terminal base of the replication origin sequence is within 200 bases.
[23] The DNA cassette according to [22], further comprising a terminator sequence located downstream of the first promoter sequence, the distance between the 3′ terminal base of the first promoter sequence and the 5′ terminal base of the terminator sequence being within 600 bases.

### Effect of the Invention

The plasmid incorporating the DNA cassette according to the present invention and the plasmid according to the present invention improve stability within host cells and increase copy number. Therefore, the plasmid production method using bacteria comprising the plasmid and the bacteria comprising the plasmid facilitates large-scale preparation of plasmids.

### BRIEF DESCRIPTION OF DRAWINGS

FIG. 1 is a staining image of bands separated by agarose electrophoresis of the RCR amplification product of plasmids obtained by incorporating various oriC cassettes into pUC4K in Example 1.

FIG. 2 is a transmitted light photograph of antibiotic-containing agar medium cultured with: transformed E. coli incorporating plasmids obtained by incorporating various oriC cassettes into pUC4K; or transformed E. coli incorporating pUC4K; in Example 1.

FIG. 3 is a graph showing the concentration of plasmids recovered from: the culture of transformed E. coli incorporating plasmids obtained by incorporating various oriC cassettes into pUC4K; or the culture of transformed E. coli incorporating pUC4K; in Example 1.

FIG. 4 is a graph showing the concentration of plasmids recovered from the culture of transformed E. coli incorporating pUC4K_T7oriC, pUC4K-PoriC_terG16, or pUC4K, in LB medium containing IPTG (FIG. 4(A)) or auto-induction medium (FIG. 4(B)) in Example 2.

FIG. 5 is a graph showing the concentration of plasmids recovered from the culture of transformed E. coli incorporating pETcoco-T7oriC (FIG. 5(A)) or pET-dnaG-PoriC_terG16 (FIG. 5(B)) in Example 3.

FIG. 6 is a graph showing the concentration of plasmids recovered from the culture of transformed E. coli incorporating pUC4K-PoriC_terG16 or pUC4K-PoriC_sg in Example 4.

FIG. 7 is a partial schematic diagram of the plasmid used in Example 5 (FIG. 7(A)) and a graph showing the concentration of plasmids recovered from the culture of transformed E. coli incorporating pUC4K, pUC4K_oriCb, or pUC4K_oriCc in Example 5 (FIG. 7(B)).

### EMBODIMENTS OF THE INVENTION

In the present invention and the specification, the term “DNA cassette” refers to double-stranded DNA with specific functions. The term “oriC cassette” refers to double-stranded DNA comprising a sequence comprising oriC, which functions in bacteria.

### DNA Cassette

In one embodiment, the DNA cassette according to the present invention comprises a replication origin sequence capable of binding to an enzyme with DnaA activity and a first promoter sequence, wherein transcription from the first promoter sequence flows into the replication origin sequence. By arranging the promoter sequence such that the 3′ side is closer to the replication origin sequence than the 5′ side and the transcription from the first promoter sequence flows into the replication origin sequence, the plasmid comprising this DNA cassette improves stability and increases copy number per host cell.

Examples of replication origin sequences capable of binding to an enzyme with DnaA activity include known replication origin sequences present in bacteria such as Escherichia coli and Bacillus subtilis, which can be obtained from public databases like NCBI. The replication origin sequence can also be obtained by cloning a DNA fragment capable of binding to an enzyme with DnaA activity and analyzing its base sequence. Modified sequences introducing mutations such as substitutions, deletions, or insertions into one or more bases of known replication origin sequences that still bind to an enzyme with DnaA activity can also be used. Preferably, the replication origin sequence used in the present invention is oriC or a modified sequence thereof, more preferably oriC derived from Escherichia coli or a modified sequence thereof.

The first promoter sequence in the DNA cassette according to the present invention may be any base sequence that can function as a promoter within the cells of the host bacteria into which the plasmid is introduced, i.e., a transcription initiation sequence capable of binding to the sigma factor of RNA polymerase or a transcription initiation sequence capable of binding to RNA polymerase derived from bacteriophages.

The first promoter sequence may be the base sequence of a wild-type promoter naturally present in any organism, a modified promoter sequence suitably altered from a wild-type promoter, or a synthetically produced promoter sequence. Preferably, the promoter sequences or modified sequences of promoters present in the genome of Escherichia coli or bacteriophages are used. Wild-type promoter sequences present in the genome of any organism can be obtained from gene sequence databases of respective organisms.

Promoters can be constitutive promoters, which induce constant expression, or inducible promoters, which can induce expression under specific culture conditions. For the first promoter sequence, either type may be used, with constitutive promoters being preferred for ease of plasmid manipulation.

Examples of the first promoter sequence include promoter sequences that can bind to sigma factors in bacteria such as Escherichia coli. Preferably, the first promoter sequence can bind to sigma factors in Escherichia coli, more preferably to sigma 70 factors. The binding of promoters to sigma factors involves sequences from the −10 region and the −35 region relative to the transcription initiation site. For example, consensus sequences that can bind to sigma 70 factors are preferably 5′-TATAAT-3′ in the −10 region and 5′-TTGACA-3′ in the −35 region. The origin of the promoter sequence is not particularly limited, but it is preferably a base sequence of various promoters commonly used in plasmids introduced into bacteria such as Escherichia coli. Specific examples include the trp promoter and lac promoter from Escherichia coli; T7, T3 and T5 promoters from bacteriophages; and synthetic promoters such as Tac promoters, and modified sequence of any of the forgoing (Non-Patent Literature 4). The term modified sequence of a promoter refers to a base sequence that retains the function of a promoter while introducing mutations such as substitutions, deletions, or insertions into one or more bases of the sequence before the modification.

In the DNA cassette according to the present invention, the distance between the replication origin sequence and the first promoter sequence is not particularly limited as long as the transcription flows into the replication origin sequence. To achieve sufficient improvements in stability and copy number of the plasmid within host cells, the distance between the 3′ terminal base of the first promoter sequence and the terminal base of the replication origin sequence is preferably within 2000 bases, more preferably within 1000 bases, even more preferably within 600 bases, and particularly preferably within 500 bases. As the replication from the replication origin sequence proceeds bidirectionally, the replication origin sequence itself can be oriented in either direction as long as the transcription from the first promoter sequence flows into it.

For ease of handling the DNA cassette, the distance between the 3′ terminal base of the first promoter sequence and the terminal base of the replication origin sequence can be within 450 bases, preferably within 400 bases, more preferably within 300 bases, even more preferably within 200 bases, and particularly preferably within 100 bases. For example, the distance between the 3′ terminal base of the first promoter sequence and the terminal base of the replication origin sequence can be within 10-80 bases, 20-80 bases, 20-70 bases, 20-60 bases, or 20-50 bases.

In one embodiment, the DNA cassette according to the present invention comprises a replication origin sequence capable of binding to an enzyme with DnaA activity, a first promoter sequence, and a gyrase-binding sequence (strong gyrase-binding sequence, SGS). SGS is a binding sequence where DNA gyrase binds and introduces negative supercoils into the DNA, further increasing the average copy number of the plasmid comprising this DNA cassette within host cells. Examples of SGS include sequences comprising the consensus sequence RNNNRNR[T/G]GRYC[G/T]YNNYNY (R=A or G, Y=C or T, N=A, G, C, or T) (SEQ ID NO: 32) or complementary sequences thereof (Non-Patent Literature 5). SGS may be derived from any species as long as it has the consensus sequence, such as phage-derived SGS or plasmid-derived SGS. SGS may also include sequences before and after the consensus sequence and can be modified sequences of SGS derived from any species. The modified sequence refers to a base sequence that retains the function of increasing the average copy number of the plasmid that comprises the modified sequence within host cells while introducing mutations such as substitutions, deletions, or insertions into one or more bases of the base sequence before the modification. SGS can be placed at any position within the DNA cassette, and the distance from the replication origin sequence and the first promoter sequence is not particularly limited. To achieve sufficient improvements in stability and copy number of the plasmid within host cells, SGS derived from pBR322 plasmid, pSC101 plasmid, or bacteriophage Mu is preferred, with SGS derived from bacteriophage Mu (Mu-SGS) (Non-Patent Literature 6) being more preferred.

In the DNA cassette comprising SGS, as long as the first promoter sequence is positioned so that transcription flows into the replication origin sequence, the position of SGS and the distance between the replication origin sequence and the first promoter sequence are not particularly limited. For example, SGS may be positioned between the replication origin sequence and a sequence selected from the first promoter sequence, ter sequence, terminator sequence, and second promoter sequence, or a sequence selected from the first promoter sequence, ter sequence, terminator sequence, and second promoter sequence may be positioned between SGS and the replication origin sequence. Particularly, since the improvement effects on the stability and copy number of the plasmid within the host cell are sufficiently obtained, the distance between the 3′ terminal base of the first promoter sequence and the terminal base of the replication origin sequence is preferably within 2000 bases, more preferably within 1000 bases.

From the viewpoint of ease of manipulation of the DNA cassette, the distance between the 3′ terminal base of the first promoter sequence and the terminal base of the replication origin sequence is preferably within 450 bases, more preferably within 400 bases, even more preferably within 300 bases, further preferably within 200 bases, and particularly preferably within 100 bases. When SGS is positioned between the 3′ terminal base of the first promoter sequence and the terminal base of the replication origin sequence, the distance between the 3′ terminal base of the first promoter sequence and the terminal base of SGS is preferably within 450 bases, more preferably within 400 bases, even more preferably within 300 bases, further preferably within 200 bases, and particularly preferably within 100 bases. For example, the distance between the 3′ terminal base of the first promoter sequence and the terminal base of the replication origin sequence or the distance between the 3′ terminal base of the first promoter sequence and the terminal base of SGS can be within 10 to 80 bases, preferably within 20 to 80 bases, more preferably within 20 to 70 bases, even more preferably within 20 to 60 bases, or within 20 to 50 bases.

The DNA cassette of the present invention may also include, in addition to the first promoter sequence and the replication origin sequence, a pair of ter sequences that are each inserted outward with respect to the replication origin sequence. Alternatively, the DNA cassette of the present invention may also include, in addition to the first promoter sequence and the replication origin sequence, a base sequence recognized by a DNA multimer separation enzyme (Patent Literature 3). This allows efficient amplification of the plasmid comprising this DNA cassette when using methods such as the RCR amplification method (Patent Literatures 1 to 3) by suppressing the production of DNA multimers.

The term “inserted outward with respect to the replication origin sequence” for ter sequences means that the ter sequences are inserted, such that replication performed in the direction of heading outside of the replication origin sequence such as oriC is allowed by the action of a combination with a protein having an activity of inhibiting replication by binding to the ter sequences, whereas replication performed in the direction of heading toward the replication origin sequence is not allowed and is terminated. Accordingly, regarding the ter sequence, the phrase “a pair of ter sequences that are each inserted outward with respect to the replication origin sequence” means a state in which one ter sequence is inserted on the 5′ side of the replication origin sequence and the other ter sequence is inserted on the 3′ side of the replication origin sequence. Examples of ter sequences inserted on the 5′ side of the replication origin sequence include sequences shown in SEQ ID NOs: 1 to 16. Examples of ter sequences inserted on the 3′ side of the replication origin sequence include sequences comprising the complementary sequence of the base sequence inserted on the 5′ side of the replication origin sequence. The position of the ter sequence is not particularly limited as long as it is inserted outward with respect to the replication origin sequence.

For example, known replication termination systems using proteins that bind to ter sequences on DNA and inhibit replication include the Tus-ter system in Escherichia coli (Non-Patent Literatures 7 and 8) and the RTP-ter system in Bacillus species (Non-Patent Literature 9). Ter sequences for these systems can be used, as well as their modified sequences. The term “modified sequence” for ter sequence refers to a base sequence in which one or more bases of the base sequence before the modification are substituted. deleted, or inserted while retaining the function as a ter sequence. When using the Tus-ter system, Tus protein is used during plasmid replication, and when using the RTP-ter system, RTP protein is used during plasmid replication.

The ter sequences included in the DNA cassette or plasmid according to the present invention may be wild-type or mutant. Wild-type ter sequences usable in the Tus-ter system include 5′-GN[A/G][T/A]GTTGTAAC[T/G]A-3′ (SEQ ID NO:1), and preferred base sequences include 5-G[T/G]A[T/A]GTTGTAAC[T/G]A-3′ (SEQ ID NO:2). 5′-GTATGTTGTAACTA-3′ (SEQ ID NO:3), 5′-AGTATGTTGTAACTAAAG-3′ (SEQ ID NO:4), 5′-GGATGTTGTAACTA-3′ (SEQ ID NO:5), 5′-GTATGTTGTAAACGA-3′ (SEQ ID NO:6), 5′-GGATGTTGTAACTA-3′ (SEQ ID NO:7), 5′-GGAAGTTGTAAACGA-3′ (SEQ ID NO:8), or 5′-GTAAAGTTGTAAACGA-3′ (SEQ ID NO:9). Mutant ter sequences are preferred for the ter sequences included in the DNA cassette or plasmid according to the present invention. Examples of the mutant ter sequences include base sequences comprising modified sequences in which one or more bases of the base sequence of SEQ ID NO:1 are substituted. Preferred mutant ter sequences include 5′-GN[A/G][T/A]GTTGTAcC[T/G]A-3′ (SEQ ID NO:10) and 5′-GTATGTTGTAcCTA-3′ (SEQ ID NO:11).

Ter sequences usable in the RTP-ter system include base sequences comprising 5′-AC[T/A][A/G]ANNNNN[C/T]NATGTACNAAAT-3′ (SEQ ID NO:12). Preferred ter sequences included in the DNA cassette for use in the RTP-ter system include base sequences comprising 5′-ACTAATT[A/G]A[A/T]C[T/C]ATGTACTAAAT-3′ (SEQ ID NO:13), 5′-ACTAATT[A/G]A[A/T]C[T/C]ATGTACTAAATTTTCA-3′ (SEQ ID NO:14), 5′-GAACTAATTAAACTATGTACTAAATTTTCA-3′ (SEQ ID NO:15), or 5′-ATACTAATTGATCCATGTACTAAATTTTCA-3′ (SEQ ID NO:16).

As long as a pair of ter sequences are each inserted outward with respect to the replication origin sequence, their arrangement is not particularly limited, and they may be positioned on the 5′ side or the 3′ side of the first promoter sequence. They may be positioned between the replication origin sequence and other sequences (e.g., SGS) or on the outside. The distance between the 3′ terminal base of the ter sequence and the terminal base of the replication origin sequence can be, for example, within 2000 bases, within 1500 bases, or within 1000 bases. In one embodiment, from the viewpoint of ease of manipulation of the DNA cassette, the distance between the 3′ terminal base of the ter sequence and the 5′ terminal base of the replication origin sequence is preferably within 300 bases, more preferably within 200 bases. The distance between the 3′ terminal base of the replication origin sequence and the 5′ terminal base of the complementary sequence of the ter sequence is preferably within 300 bases, more preferably within 200 bases.

From the viewpoint of suppressing the production of DNA multimers and efficiently obtaining the amplification product of the plasmid when amplifying in vitro using methods such as the RCR amplification method, the distance between the 3′ terminal base of the ter sequence and the 5′ terminal base of the replication origin sequence or the distance between the 3′ terminal base of the replication origin sequence and the 5′ terminal base of the complementary sequence of the ter sequence is preferably within 300 bases, more preferably within 200 bases. For example, the distance between the 3′ terminal base of the ter sequence and the 5′ terminal base of the replication origin sequence or the distance between the 3′ terminal base of the replication origin sequence and the 5′ terminal base of the complementary sequence of the ter sequence can be within 10 to 80 bases, preferably within 20 to 80 bases, more preferably within 20 to 70 bases, even more preferably within 20 to 60 bases, or within 20 to 50 bases. When SGS is positioned adjacent to the replication origin sequence, the distance between the 3′ terminal base of the ter sequence and the 5′ terminal base of the replication origin sequence or SGS, or the distance between the 3′ terminal base of the replication origin sequence or SGS and the 5′ terminal base of the complementary sequence of the ter sequence is preferably within 300 bases, more preferably within 200 bases, for example, within 10 to 80 bases, 20 to 80 bases, 20 to 70 bases, 20 to 60 bases, or 20 to 50 bases.

The DNA cassette of the present invention may include a terminator sequence downstream of the first promoter sequence. In the DNA cassette of the present invention, it is preferred that the terminator sequence is positioned on the 3′ side of the first promoter sequence and further on the 3′ side of the replication origin sequence. The distance between the 3′ terminal base of the first promoter sequence and the 5′ terminal base of the terminator sequence is not particularly limited, but it can be within 2500 bases, preferably within 2000 bases, more preferably within 1000 bases, and even more preferably within 600 bases.

When the terminator sequence is positioned 3′ side of the first promoter sequence and further 3′ side of the replication origin sequence, the distance between the 3′ terminal base of the replication origin sequence and the 5′ terminal base of the terminator sequence is not particularly limited, but it can be within 2000 bases, within 1500 bases, or within 1000 bases. In one embodiment, from the viewpoint of ease of manipulation of the DNA cassette, the distance is preferably within 300 bases, more preferably within 200 bases. For example, the distance between the 3′ terminal base of the ter sequence and the 5′ terminal base of the terminator sequence can be within 10 to 80 bases, preferably within 20 to 80 bases, more preferably within 20 to 70 bases, even more preferably within 20 to 60 bases, or within 20 to 50 bases.

The terminator sequence included in the DNA cassette of the present invention is not particularly limited as long as it is a base sequence that can function as a terminator within the cells of the host bacteria into which the plasmid comprising this DNA cassette is introduced, i.e., a base sequence capable of terminating transcription.

The terminator sequence can be a base sequence of a terminator (wild-type terminator) originally possessed by any organism, a base sequence of a terminator (mutant terminator) appropriately modified from a wild-type terminator, or a base sequence of a terminator artificially synthesized. Among them, the base sequence of the terminator of each gene existing in the genome of Escherichia coli or bacteriophages and its modified sequences are preferred. The terminator sequence of bacteria can be either rho-dependent or rho-independent. Examples of bacterial terminator sequences include the terminator sequence of the formate dehydrogenase gene (fdhF) and the T1 or T2 terminator (particularly the T1 terminator) of ribosomal RNA genes (rrnB, etc.) of Escherichia coli. Wild-type terminator sequences possessed by the genome of any organism can be obtained from the genetic sequence databases of each organism.

In one embodiment, the present invention also relates to a DNA cassette comprising a replication origin sequence capable of binding to an enzyme with DnaA activity, a first promoter sequence, and a terminator sequence, wherein transcription from the first promoter sequence flows into the replication origin sequence and the terminator sequence is located downstream of the first promoter sequence. In this DNA cassette, the distance between the 3 terminal base of the first promoter sequence and the terminal base of the replication origin sequence is within 200 bases, and the distance between the 3′ terminal base of the first promoter sequence and the 5′ terminal base of the terminator sequence is within 600 bases, for example, between 300 and 600 bases. This DNA cassette may further include a pair of ter sequences that are each inserted outward with respect to the replication origin sequence. This DNA cassette may have the terminator sequence located downstream of the first promoter sequence and also downstream of the replication origin sequence. This DNA cassette may include the replication origin sequence with a pair of ter sequences inserted that are each inserted outward with respect to the replication origin sequence and further have the terminator sequence located downstream of the replication origin sequence. In one embodiment, the length of this DNA cassette is between 300 and 2000 base pairs (bp), preferably between 350 and 1000 bp, and more preferably between 400 and 1000 bp.

In one embodiment, the present invention also provides a DNA cassette comprising a replication origin sequence capable of binding to an enzyme with DnaA activity, a first promoter sequence, and a pair of ter sequences that are each inserted outward with respect to inserted outwardly relative to the replication origin sequence, wherein transcription from the first promoter sequence flows into the replication origin sequence, and the distance between the 3′ terminal base of the first promoter sequence and the terminal base of the replication origin sequence is within 200 bases. The DNA cassette may further include a terminator sequence located downstream of the first promoter sequence, and the distance between the 3′ terminal base of the first promoter sequence and the 5′ terminal base of the terminator sequence may be within 600 bases. The position of the pair of ter sequences that are each inserted outward with respect to the replication origin sequence within the DNA cassette is not particularly limited. In one embodiment, the DNA cassette includes the replication origin sequence with the pair of ter sequences that are each inserted outward with respect to it, located downstream of the first promoter sequence. The distance between the 3′ terminal base of the ter sequence and the replication origin sequence or the distance between the replication origin sequence and the 5′ terminal base of the complementary sequence of the ter sequence is preferably within 300 bases, and more preferably within 200 bases. For example, the distance can be within 10 to 80 bases, within 20 to 80 bases, within 20 to 70 bases, within 20 to 60 bases, or within 20 to 50 bases. In one embodiment, the length of the DNA cassette is 300 to 2000 base pairs (bp), preferably 350 to 1000 bp, more preferably 400 to 1000 bp.

The DNA cassette of the present invention preferably further includes a complementary sequence of a second promoter sequence on the 3′ side of the first promoter sequence and on the 5′ side of the replication origin sequence. By inserting the second promoter sequence in the direction that causes head-on collision (reverse direction) with the first promoter sequence and cancelling the effect of transcription flowing into the replication origin sequence by reverse direction transcription from the second promoter, the stability of the plasmid comprising the DNA cassette within the host cell can be further improved, and the average copy number per host cell can be increased.

The position of the second promoter sequence within the DNA cassette is not particularly limited as long as it is on the 3′ side of the first promoter sequence and on the 5′ side of the replication origin sequence. For example, the first promoter sequence and the complementary sequence of the second promoter sequence may be close to each other, or the first promoter sequence and the replication origin sequence may be close to each other.

As the second promoter sequence, the same as the promoter sequences listed for the first promoter sequence can be used. It is preferable to use a promoter sequence different from the first promoter sequence, and an inducible promoter is more preferred. In one embodiment, a constitutive promoter can be used as the first promoter sequence, and an inducible promoter can be used as the second promoter sequence.

### Plasmid

The present invention also relates to a plasmid comprising: a replication origin sequence capable of binding to an enzyme with DnaA activity; a first promoter sequence; and a plasmid replication origin; wherein transcription from the first promoter sequence flows into the replication origin sequence, and the distance between the 3′ terminal base of the first promoter sequence and the terminal base of the replication origin sequence is within 2000 bases. In this plasmid, for example, the distance between the 3′ terminal base of the first promoter sequence and the terminal base of the replication origin sequence is within 1000 bases, preferably within 600 bases, more preferably within 500 bases, even more preferably within 400 bases, and particularly preferably within 300bases. The plasmid replication origin in this plasmid is preferably of the ColEl type, similar to the DNA cassette of the present invention. The plasmid may also further include the complementary sequence of the second promoter sequence on the 3′ side of the first promoter sequence and on the 5′ side of the replication origin sequence. The plasmid may also further include a SGS, a pair of ter sequences that are each inserted outward with respect to the replication origin sequence, and a terminator sequence downstream of the first promoter sequence. The present invention also relates to a plasmid obtained by introducing the DNA cassette of the present invention into a plasmid comprising a plasmid replication origin. These plasmids of the present invention have improved stability and average copy number within the host cell. Therefore, the plasmid of the present invention is preferably a plasmid introduced into host cells in which the replication origin sequence of the plasmid can function.

Examples of plasmid replication origins included in the plasmid of the present invention include ColE1 type, p15A type, pSC101 type, P1 type, F type, R1 type, R6Kγ type, λ type, φB2 type, φB0 type, RK2 type, P4 type, and other plasmid replication origins functioning in Escherichia coli, with ColE1 type being particularly preferred due to its high copy number. ColE1 type replication origins include: pMB1 which is a replication origin of plasmids such as pUC, pGEM, pTZ and pBR322; ColE1 which is a replication origin of plasmids such as pBluescript, or modified sequences thereof. The modified sequences refer to base sequences in which one or more bases of the base sequence before the modification are substituted, deleted, or inserted while retaining the function as a plasmid replication origin.

Introduction of the DNA cassette or individual sequences into the plasmid can generally be carried out using various gene modification techniques used when incorporating DNA fragments into circular DNA. For example, by providing restriction enzyme sites of the same type as those possessed by the target plasmid at both ends of the DNA cassette, and linking the digested DNA cassette fragment and plasmid fragment by ligation, the DNA cassette can be introduced into the plasmid. The plasmid can also be linearized, linked with the DNA cassette fragment, and then circularized. The same applies to the introduction of individual sequences. In a plasmid comprising a plasmid replication origin and the promoters described above, the replication origin sequence can be introduced so that transcription from the promoter flows into the replication origin sequence to obtain the plasmid of the present invention. The linearization of the plasmid can be performed by restriction enzyme treatment or by PCR amplification using the plasmid as a template. Linking reactions of linear DNA fragments include methods such as the In-Fusion method (Patent Literature 4), Gibson Assembly method (Patent Literatures 5 and 6), and Recombination Assembly method (Patent Literature 7).

As a method for directly incorporating the DNA cassette fragment into a circular plasmid, for example, Gateway cloning using a site-specific recombination mechanism is known (Non-Patent Literature 10), and reagent kits are commercially available (Thermo Fisher). Gateway cloning requires that the circular DNA targeted for the insertion of linear DNA fragments has recombination sequences recognized by site-specific recombinase. Other methods for inserting or replacing linear DNA fragments into circular DNA in cells include the Recombineering method using a homologous recombination mechanism (Non-Patent Literature 11). Additionally, as shown in the following examples, DNA cassette fragments can be incorporated into plasmids by adding base sequences homologous to the target regions in the plasmid at both ends of the DNA cassette fragment and performing homologous recombination reactions in vitro using RecA family recombinase and, if necessary, exonuclease. The OriCiro Cell-Free Switching System (OriCiro Genomics) can be used for this reaction.

In one embodiment, the present invention provides a method for preparing a plasmid comprising a DNA cassette, the method comprising providing a DNA cassette comprising a replication origin sequence capable of binding to an enzyme with DnaA activity and introducing the DNA cassette into a plasmid comprising a plasmid replication origin, wherein introducing the DNA cassette into the plasmid comprises preparing a reaction solution comprising the plasmid, the DNA cassette, a protein with RecA family recombinase activity, and an exonuclease, and incubating the reaction solution to perform a homologous recombination reaction. In the method, the plasmid contains regions Ha and Hb, the region Hb being located downstream of the region Ha, and the DNA cassette contains a homologous region corresponding to the region Ha and a homologous region corresponding to the region Hb, the latter being positioned downstream of the former.

The homologous recombination reaction can be carried out with reference to the examples described later. Briefly, a DNA cassette fragment with homologous sequences to regions Ha and Hb on both ends that flank the target region to be introduced into the plasmid (i.e., a DNA cassette fragment with a homologous region corresponding to region Ha and a homologous region corresponding to region Hb on both ends) is prepared. A reaction solution comprising the DNA cassette, the plasmid, RecA family recombinase, and exonuclease is prepared, and this solution is incubated to perform the homologous recombination reaction. The reaction temperature is preferably within the range of 20 to 48° C., more preferably within the range of 24 to 42° C.

The RecA family recombinase refers to proteins that polymerize on single-stranded or double-stranded DNA to form filaments, have hydrolytic activity against nucleoside triphosphates such as ATP (adenosine triphosphate), and have the function (RecA family recombinase activity) of searching for homologous regions and performing homologous recombination. RecA family recombinase proteins include prokaryotic RecA homologs (such as Escherichia coli RecA), bacteriophage RecA homologs, archaeal RecA homologs, and eukaryotic RecA homologs. These can be wild-type proteins or modified proteins retaining RecA family recombinase activity with mutations that delete, add, or substitute 1 to 30, preferably 1 to 10, more preferably 1 to 5 amino acids in the wild-type protein. The amount of RecA family recombinase protein in the reaction solution is not particularly limited, but at the start of the reaction, it is preferably within the range of 0.01 to 100 μM, more preferably within the range of 0.1 to 100 μM, even more preferably within the range of 0.1 to 50 μM, further more preferably within the range of 0.5 to 10 μM, and particularly preferably within the range of 1.0 to 5.0 μM.

The exonuclease can be any enzyme with the activity to sequentially hydrolyze from the 3′ end or the 5′ end of linear DNA, regardless of its type or biological origin. For example, preferred 3′→5′ exonucleases include AP endonucleases of the exonuclease III family type such as exonuclease III, and preferred 5′→3′ exonucleases include T5 exonuclease.

The base pair length of regions Ha and Hb, and the homologous regions corresponding to regions Ha and Hb, is preferably 10 base pairs or more, more preferably 15 base pairs or more, and even more preferably 20 base pairs or more. The base pair length of regions Ha and Hb is preferably 500 base pairs or less, more preferably 300 base pairs or less, even more preferably 200 base pairs or less, and further more preferably 150 base pairs or less.

The amount of plasmid and DNA cassette in the reaction solution is not particularly limited, but at the start of the reaction, it is preferably 0.4 pM or more, more preferably 4 pM or more, and even more preferably 40 pM or more. For higher homologous recombination efficiency, the total concentration of plasmid and DNA cassette in the reaction solution at the start of the reaction is preferably 100 nM or less, more preferably 40 nM or less, even more preferably 4 nM or less, and particularly preferably 0.4 nM or less.

The reaction solution preferably contains at least one of nucleoside triphosphates (selected from ATP, GTP, CTP, UTP, and m5UTP) and deoxynucleotide triphosphates (selected from dATP, dGTP, dCTP, and dTTP), and a magnesium ion (Mg²⁺) source (such as Mg(OAc)₂, MgCl₂, MgSO4). It preferably further includes a combination of a regenerating enzyme for nucleoside triphosphates or deoxynucleotide triphosphates and its substrate (such as a combination of: creatine kinase and creatine phosphate; pyruvate kinase and phosphoenolpyruvate; acetate kinase and acetyl phosphate; polyphosphate kinase and polyphosphate; and nucleoside diphosphate kinase and nucleoside triphosphate).

Preferred host cells into which the plasmid of the present invention is introduced include bacteria such as Escherichia coli, Bacillus subtilis, Actinomycetes, and archaea, with Escherichia coli or Actinomycetes being more preferred, and Escherichia coli being particularly preferred as it is widely used for large-scale preparation of plasmids. The plasmid into which the DNA cassette of the present invention is incorporated is not particularly limited, and by incorporating the DNA cassette of the present invention into any known plasmid, the copy number of plasmid maintained per host cell can be increased, and the plasmid can be more stably maintained within the host cell. Preferred plasmids include those having the plasmid replication origins described above, particularly pUC, pBR322, pBluescript, pGEM, or pTZ plasmids having ColE1 type replication origins such as ColE1 or pMB1. More preferred plasmids include pUC, and particularly preferred are high-copy plasmids such as pUC18, pUC19, pUC57, pBluescript, and their derivative plasmids, which have an average copy number of 500 to 700 or more per host cell. By incorporating the DNA cassette of the present invention into such high-copy plasmids, their copy number can be further increased. In one embodiment, the plasmid of the present invention, when functioning within the host cell (preferably Escherichia coli), has an average copy number of 1 copy or more, preferably 10 or more, more preferably 20 or more, even more preferably 20 to 10,000, and particularly preferably 500 to 5,000 per host cell. Additionally, the plasmid can be selected according to the characteristics of the promoter used.

The introduction of plasmids comprising the DNA cassette of the present invention into host cells can be carried out using various methods commonly used for introducing plasmids into bacterial cells or their modified methods. Methods for introducing plasmids include PEG (polyethylene glycol) methods, chemical methods, and electroporation methods. These methods can be carried out according to standard procedures.

The DNA cassette of the present invention contains a replication origin sequence capable of binding to an enzyme with DnaA activity. The plasmid comprising this cassette can be amplified in vitro using the RCR amplification method.

### Method for Preparing Plasmid

In one embodiment, the present invention provides a method for preparing a plasmid comprising providing a DNA cassette comprising a replication origin sequence capable of binding to an enzyme with DnaA activity and a first promoter sequence, and introducing the DNA cassette into a plasmid comprising a plasmid replication origin. The DNA cassette contains a replication origin sequence capable of binding to an enzyme with DnaA activity and a first promoter sequence, and transcription from the first promoter sequence flows into the replication origin sequence, with the distance between the 3′ terminal base of the first promoter sequence and the terminal base of the replication origin sequence being within 2000 bases. In one embodiment, the prepared plasmid is a high-copy plasmid with a higher average copy number per host cell (preferably Escherichia coli) compared to the plasmid used for its preparation, under the same conditions.

In this method, as long as the DNA cassette to be introduced into the plasmid has a first promoter sequence positioned to direct transcription into the replication origin sequence, the distance between the replication origin sequence and the first promoter sequence is not particularly limited. For example, the distance between the 3′ terminal base of the first promoter sequence and the terminal base of the replication origin sequence can be within 1000 bases, preferably within 600 bases, more preferably within 500 bases, even more preferably within 400 bases, and particularly preferably within 300 bases. The plasmid replication origin in this method is preferably of the ColE1 type, similar to the DNA cassette of the present invention. The DNA cassette in this method, similar to the DNA cassette of the present invention, may further include the complementary sequence of the second promoter sequence on the 3′ side of the first promoter sequence and on the 5′ side of the replication origin sequence, a SGS, a pair of ter sequences that are each inserted outward with respect to the replication origin sequence, and a terminator sequence downstream of the first promoter sequence.

As described above, the plasmid of the present invention can be obtained by introducing the replication origin sequence into a plasmid comprising a plasmid replication origin and promoters in a way that transcription from the promoter flows into the replication origin sequence. Therefore, in one embodiment, the present invention also provides a method for preparing a plasmid comprising providing a DNA cassette comprising a replication origin sequence capable of binding to an enzyme with DnaA activity, providing a plasmid comprising a plasmid replication origin and a promoter sequence, and introducing the DNA cassette into the plasmid in a way that transcription from the promoter sequence of the plasmid flows into the replication origin sequence. By introducing the replication origin sequence into the plasmid in a way that the 3′ side of the promoter sequence is closer to the replication origin sequence than the 5′ side of the promoter sequence, transcription from the promoter sequence of the plasmid can flow into the replication origin sequence. In this method, the DNA cassette may further contain one or more sequences selected from gyrase-binding sequences, second promoter sequences, and terminator sequences. The method may further include introducing a DNA cassette comprising one or more sequences selected from gyrase-binding sequences, second promoter sequences, and terminator sequences into the plasmid. In one embodiment, the present invention also provides a method for preparing a plasmid comprising a DNA cassette comprising providing a DNA cassette comprising a replication origin sequence capable of binding to an enzyme with DnaA activity and a gyrase-binding sequence, providing a plasmid comprising a plasmid replication origin and a promoter sequence, and introducing the DNA cassette into the plasmid in a way that transcription from the promoter sequence of the plasmid flows into the replication origin sequence.

The prepared plasmid is preferably a high-copy plasmid with a higher average copy number per host cell (preferably Escherichia coli) under the same conditions compared to the plasmid used for its preparation. Each step can be carried out with reference to the descriptions of the DNA cassette, plasmid, and method for preparing a plasmid of the present invention. In one embodiment, the distance between the 3′ terminal base of the promoter sequence and the terminal base of the replication origin sequence is within 2000 bases, preferably within 1000 bases, more preferably within 600 bases, even more preferably within 500 bases.

Furthermore, in one embodiment, the present invention also provides a DNA cassette for preparing a high-copy plasmid comprising a replication origin sequence capable of binding to an enzyme with DnaA activity and a gyrase-binding sequence. This DNA cassette may further contain one or more sequences selected from second promoter sequences and terminator sequences. By introducing this DNA cassette into a plasmid comprising a plasmid replication origin and a promoter sequence in a way that transcription from the promoter sequence of the plasmid flows into the replication origin sequence, a high-copy plasmid can be prepared. The length of the DNA cassette for preparing a high-copy plasmid is not particularly limited, but for example, it is within 300 to 2000 bases, preferably within 1000 bases, more preferably within 600 bases, even more preferably within 500 bases.

### Method for Producing Plasmid

The plasmid comprising the DNA cassette of the present invention or the plasmid of the present invention can be introduced into host cells such as Escherichia coli, and the resulting transformants can be cultured and proliferated to replicate the plasmid within the host cells. By extracting the replicated plasmid from the culture of the host cells and purifying it as needed, the plasmid can be produced in large quantities. The culture of the transformants and the recovery of the plasmid from the transformants can be performed using general methods used for culturing bacteria such as Escherichia coli and extracting DNA.

If the promoter in the DNA cassette or plasmid of the present invention is an inducible promoter, the host cells are cultured under specific conditions that induce the expression of the inducible promoter. This further improves the stability of the plasmid within the host cells and increases the copy number per host cell, enabling the recovery of a larger quantity of plasmid.

The plasmid comprising the DNA cassette of the present invention and the plasmid of the present invention can be used in genetic engineering research and as raw materials for gene therapy. For example, the plasmid can be used as a vector for virus production. Additionally, the plasmid comprising the DNA cassette of the present invention or the plasmid of the present invention can be used as raw materials to produce single-stranded RNA such as mRNA. The production of RNA can be performed using general methods used for RNA transcription from plasmid DNA.

### EXAMPLES

The present invention will be further described in detail with reference to the following examples, but the invention is not limited to these examples.

### Example 1

Three types of DNA fragments containing oriC were prepared and incorporated into the plasmid pUC4K (GenBank Accession No.: X06404, 3.9 kbp), which does not have an oriC sequence. Transformants of Escherichia coli were prepared with these plasmids, and the growth and replication of the plasmids by the transformants were examined.

### Preparation of oriC Cassette Fragments

A DNA fragment was prepared as an oriC_ter cassette (378 bp, SEQ ID NO: 19), where a wild-type ter sequence (terWT sequence, SEQ ID NO: 18) is connected adjacent to the 5′ side of the oriC sequence (SEQ ID NO: 17) derived from the Escherichia coli chromosome, and the complementary sequence of terWT sequence is connected adjacent to the 3′ side of the oriC. Both ends of the fragment have overlapping sequences homologous to the adjacent 40 bp regions outside the pUC origin in pUC4K. The terWT sequence includes the base sequence of SEQ ID NO: 4.

A DNA fragment was prepared as a PoriC_terG16 cassette (426 bp, SEQ ID NO: 24), where a mutant ter sequence (terG16 sequence, SEQ ID NO: 21) is connected adjacent to the 5′ side of the oriC sequence (SEQ ID NO: 20) derived from the Escherichia coli chromosome, and the complementary sequence of terG16 sequence is connected near the 3′ side of the oriC. Additionally, the Tac promoter sequence (SEQ ID NO: 22) is positioned near the 5′ side of terG16 sequence, and the terminator sequence of the Escherichia coli fdhF gene (SEQ ID NO: 23) is positioned near the 3′ side of the complementary sequence of terG16. The terG16 sequence (SEQ ID NO: 11) includes a one-base mutation from the sequence of SEQ ID NO: 4.

A DNA fragment was prepared as a PoriC cassette (390 bp, SEQ ID NO: 25) by removing the terG16 sequence on the 5′ side of oriC and the complementary sequence of terG16 sequence on the 3′ side of oriC from the PoriC_terG16 cassette.

The base sequences of each oriC cassette are shown in Table 1. In the table, the region indicated by uppercase letters at the 5′ end represents the Tac promoter sequence, the region indicated by uppercase letters at the 3′ end represents the fdhF terminator sequence, and the region indicated by uppercase letters near the center represents the oriC sequence. The regions enclosed in squares represent the terWT sequence, terG16 sequence, and their complementary sequences. The lowercase regions at the 5′ and 3′ ends of the oriC_ter cassette represent the overlapping sequences.

TABLE 1

| | | SEQ |
|---|---|---|
| | | ID |
| cassette | Base sequence | NO: |
| oriC_ter | 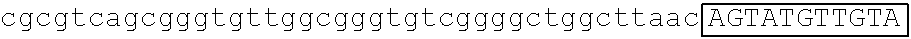 | 19 |
| | 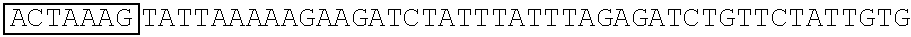 | |
| | ATCTCTTATTAGGATCGCACTGCCCTGTGGATAACAAGGATCCGGCTTTTAA | |
| | GATCAACAACCTGGAAAGGATCATTAACTGTGAATGATCGGTGATCCTGGAC | |
| | CGTATAAGCTGGGATCAGAATGAGGGGTTATACACAACTCAAAAACTGAACA | |
| | ACAGTTGTTCTTTGGATAACTACCGGTTGATCCAAGCTTCCTGACAGAGTTA | |
| | 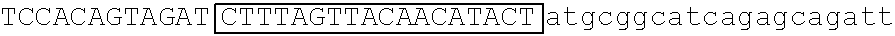 | |
| | gtactgagagtgcaccat | |
| PoriC_terG16 | GAGCTGTTGACAGAGGGTCATTTTCACACTATAATGcagtgaatcccaaac | 24 |
| | 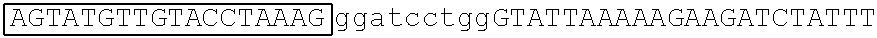 | |
| | ATTTAGAGATCTGTTCTATTGTGATCTCTTATTAGGATCGCACTGCCCTGTG | |
| | GATAACAAGGATCCGGCTTTTAAGATCAACAACCTGGAAAGGATCATTAACT | |
| | GTGAATGATCGGTGATCCTGGACCGTATAAGCTGGGATCAGAATGAGGGGTT | |
| | ATACACAACTCAAAAACTGAACAACAGTTGTTCTTTGGATAACTACCGGTTG | |
| | ATCCAAGCTTCCTGACAGAGTTATCCACAGTAGATcgcacgatctgtcagct | |
| | 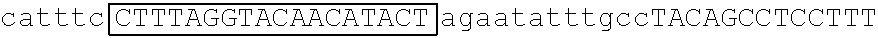 | |
| | CGGAGGCTGTTTTTTTA | |
| PoriC | GAGCTGTTGACAGAGGGTCATTTTCACACTATAATGcagtgaatcccaaac | 25 |
| | ggatcctggGTATTAAAAAGAAGATCTATTTATTTAGAGATCTGTTCTATT | |
| | GTGATCTCTTATTAGGATCGCACTGCCCTGTGGATAACAAGGATCCGGCTTT | |
| | TAAGATCAACAACCTGGAAAGGATCATTAACTGTGAATGATCGGTGATCCTG | |
| | GACCGTATAAGCTGGGATCAGAATGAGGGGTTATACACAACTCAAAAACTGA | |
| | ACAACAGTTGTTCTTTGGATAACTACCGGTTGATCCAAGCTTCCTGACAGAG | |
| | TTATCCACAGTAGATcgcacgatctgtcagctcatttcagaatatttgccT | |
| | ACAGCCTCCTTTCGGAGGCTGTTTTTTTA | |

### Incorporation of oriC Cassette Fragments into pUC4K

Using the OriCiro Cell-Free Switching System, which utilizes a homologous recombination reaction, each oriC cassette fragment was incorporated into pUC4K. The target region in pUC4K was a 60 bp region common to ColE1-type plasmids, adjacent to the outside of the ColE1 origin (native oriC in the ColE1-type plasmids). First, using each oriC cassette fragment as a template, PCR was performed with the primer sets listed in Table 2 to obtain DNA fragments as amplification products with overlapping sequences homologous to the target region (60 bp) at both ends. In the base sequences of each primer in Table 2, the regions indicated by uppercase letters represent the overlapping sequences.

TABLE 2

| | | SEQ |
|---|---|---|
| | | ID |
| Primer | Base sequence | NO: |
| ColE1_ | TGACCAAAATCCCTTAACGTGAGTTTT | 26 |
| Fw | CGTTCCACTGAGCGTCAGACCCCGTAG | |
| | AAAAGAgagctgttgacagagggtc | |
| ColE1_ | TTTTTGTTTGCAAGCAGCAGATTACGC | 27 |
| Rv | GCAGAAAAAAAGGATCTCAAGAAGATC | |
| | CTTTGAtaaaaaaacagcctccgaaag | |
| | gag | |

The reaction of the OriCiro Cell-Free Switching System was performed using 200 pM of pUC4K and 200 pM of the oriC cassette fragment with added overlapping sequences. After performing the RCR amplification reaction according to the kit manual, 1 μL of the dilution solution was subjected to agarose electrophoresis, and the separated bands were stained with SYBR Green.

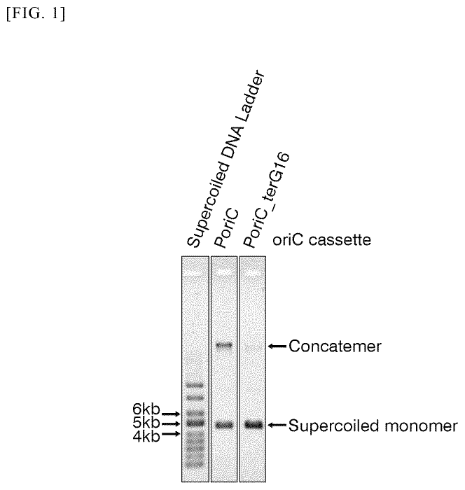

The staining results are shown in FIG. 1. In the figure, the lane labeled “PoriC” shows the RCR amplification product of the plasmid pUC4K-PoriC, which was prepared by incorporating the PoriC cassette into pUC4K. The lane labeled “PoriC_terG16” shows the RCR amplification product of the plasmid pUC4K-PoriC_terG16, which was prepared by incorporating the PoriC_terG16 cassette into pUC4K. As shown in FIG. 1, in the case of pUC4K-PoriC, which does not contain the terG16 sequence, concatemer products were also secondarily amplified. In contrast, in the case of pUC4K-PoriC_terG16, the amplification of concatemers was suppressed, and the plasmid was amplified as a monomeric supercoiled form. These results indicate that the PoriC_terG16 cassette functions as an origin of replication for RCR and that the terG16 sequence, which has a base substitution in the consensus sequence of ter, also functions to suppress concatemers.

### Transformants with Plasmids Containing Incorporated oriC Cassettes

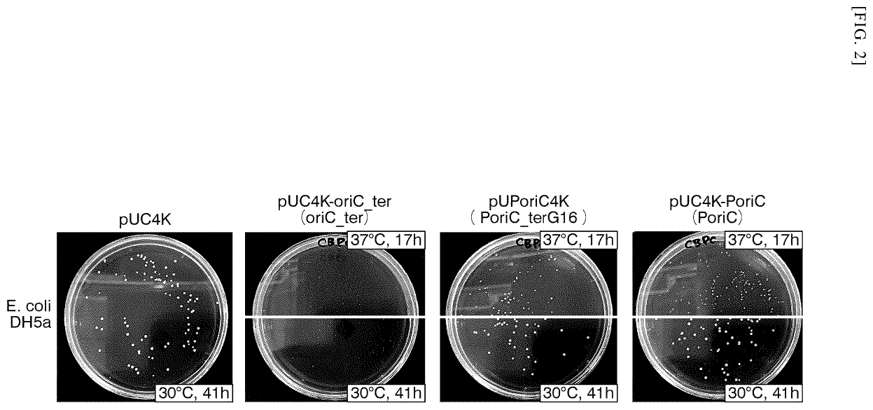

Each oriC cassette was incorporated into pUC4K to prepare plasmids. These plasmids and pUC4K (10 ng) was introduced into Escherichia coli DH5α cells (Takara Bio) by chemical transformation. A portion of the transformants was then cultured on LB agar plates containing 100 μg/mL carbenicillin at 30° C. for 41 hours. Transmitted light photographs of the cultured agar plates are shown in FIG. 2.

As shown in FIG. 2, when the plasmid pUC4K-oriC_ter, which incorporates the oriC_ter cassette into pUC4K, was introduced, the resulting colonies were significantly smaller compared to those with pUC4K, and the growth of the transformed E. coli was inhibited. In contrast, in transformants with pUC4K-PoriC and pUC4K-PoriC_terG16, which incorporate oriC cassettes with a promoter sequence positioned to direct transcription into oriC, colony formation was observed at a size comparable to that with pUC4K. No growth differences were detected based on the presence of the terG16 sequence near oriC. These results confirm that incorporating an oriC cassette with a promoter sequence positioned to direct transcription into oriC improves the stability of the plasmid in Escherichia coli and allows transformants containing the plasmid to grow almost normally.

The copy number of pUC4K-PoriC_terG16 within the cells of transformed E. coli containing pUC4K-PoriC_terG16 was compared to the copy number of pUC4K within the cells of transformed E. coli containing pUC4K. Specifically, multiple colonies of E. coli were selected from the agar plates, and each transformant was cultured in 10 mL of liquid medium containing 50 μg/mL carbenicillin at 37° C. for 16 hours with shaking. LB or 2×YT liquid medium was used as the liquid medium. After culturing, the turbidity of the culture broth at 600 nm (A600) was measured, and a volume equivalent to [turbidity (A600)]×[volume (mL)]=3 was taken to equalize the number of E. coli cells in the sample used for plasmid extraction. Plasmid extraction was performed using a commercial DNA extraction kit (product name: QIAprep Spin Miniprep Kit, QIAGEN). The concentration of the plasmid DNA solution eluted with 50 μL of 10 mM Tris-Cl (pH 8.5) was measured using a fluorometer (product name: Quantus (Registered Trademark) Fluorometer, Promega).

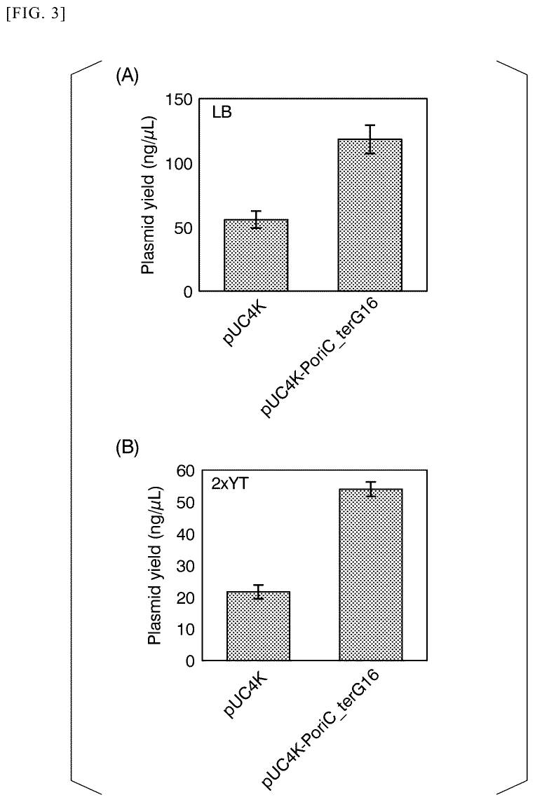

The results of the plasmid concentration measurements of the obtained plasmid DNA solution are shown in FIG. 3. FIG. 3(A) shows the results of culturing in LB medium, and FIG. 3(B) shows the results of culturing in 2×YT medium. The plasmid extraction was performed from five independent colonies in LB medium and from three independent colonies in 2×YT medium, with the average values and standard errors shown. Additionally, there was no significant difference in the turbidity of the cultures between transformants containing pUC4K-PoriC and those containing pUC4K, confirming that there was no difference in the growth of both transformants.

As shown in FIG. 3, in both LB and 2×YT media, the amount of plasmid recovered from transformants containing pUC4K-PoriC_terG16 was more than double that recovered from transformants containing pUC4K. These results confirm that incorporating the PoriC_terG16 cassette into pUC4K increased the copy number per Escherichia coli cell by approximately 2.5 times. Since the copy number of pUC4K is 500 to 700, the copy number of pUC4K-PoriC_terG16 was estimated to have increased to about 1000 to 1800. Additionally, similar copy number measurements were performed using pUC4K-oriC_ter, which incorporates the PoriC_ter cassette with a wild-type ter sequence, and it was found that while the copy number per E. coli cell increased compared to pUC4K, the ability to increase the copy number was lower than that of pUC4K-PoriC_terG16.

### Example 2

A T7oriC cassette was constructed by inserting an inducible T7 promoter in the direction of a head-on collision with the Tac promoter sequence on the 3′ side of the Tac promoter sequence and the 5′ side of the terG16 sequence in the PoriC_terG16 cassette prepared in Example 1, to counteract the effect of transcription flowing into oriC from the T7 promoter. The effect of the insertion of this T7oriC cassette (in other words, the effect of the transcription induction from the T7 promoter) on the copy number of pUC plasmid was examined. In the base sequence of the T7oriC cassette shown in Table 3, the underlined regions represent the complementary sequence of the T7 promoter sequence.

TABLE 3

| | | SEQ |
|---|---|---|
| | | ID |
| cassette | Base sequence | NO: |
| T7oriC | GAGCTGTTGACAGAGGGTCATTTTCACACTATAATGCCGCTATAGTGAGTCGT | 28 |
| | 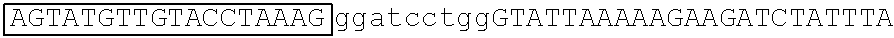 | |
| | TTTAGAGATCTGTTCTATTGTGATCTCTTATTAGGATCGCACTGCCCTGTGGAT | |
| | AACAAGGATCCGGCTTTTAAGATCAACAACCTGGAAAGGATCATTAACTGTGAA | |
| | TGATCGGTGATCCTGGACCGTATAAGCTGGGATCAGAATGAGGGGTTATACACA | |
| | ACTCAAAAACTGAACAACAGTIGTTCTTTGGATAACTACCGGTTGATCCAAGCT | |
| | 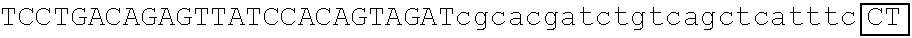 | |
| | 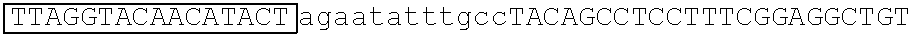 | |
| | TTTTTTA | |

A DNA fragment consisting of the T7oriC cassette was prepared in the same manner as in Example 1 and incorporated into pUC4K to prepare the plasmid pUC4K_T7oriC.

Next, since transcription from the T7 promoter requires T7 phage RNA polymerase (T7 RNAP), pUC4K_T7oriC or pUC4K was introduced into Escherichia coli NovaBlue (DE3) cells (Novagen), which express T7 RNAP in an IPTG-dependent manner, to obtain transformants. The obtained transformants were cultured in 10 mL of antibiotic-containing LB medium (50 μg/mL carbenicillin) (-IPTG) or antibiotic-containing LB medium added with 1 mM IPTG (+IPTG) at 37° C. for 16 hours.

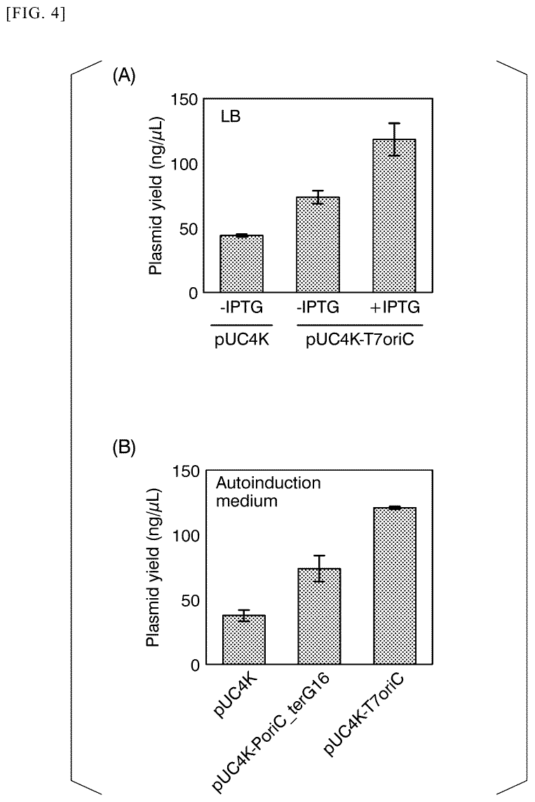

In the same manner as in Example 1, plasmid extraction was performed from the cultures after culturing while equalizing the number of the cells, and the concentration of the obtained plasmid DNA solution was measured. The measurement results are shown in FIG. 4(A). Plasmid extraction was performed from two independent colonies, with the average values and standard errors shown.

Additionally, transformants obtained by introducing pUC4K_T7oriC, pUC4K_PoriC_terG16, or pUC4K were cultured using auto-induction medium for Escherichia coli culture systems, where lactose induction occurs automatically according to the growth phase of Escherichia coli, instead of IPTG. The auto-induction medium (product name: MagicMedia E. coli Expression Medium, Thermo Fisher Scientific) was used for the culture and cultured in the same manner, and plasmid extraction was performed from the cultures after culturing while equalizing the number of the cells, followed by measurement of the concentration of the obtained plasmid DNA solution. The measurement results are shown in FIG. 4(B). Plasmid extraction was performed from two independent colonies, with the average values and standard errors shown.

As shown in FIG. 4(A), in the absence of IPTG induction, the amount of plasmid recovered from transformants containing pUC4K_T7oriC was higher than that recovered from transformants containing pUC4K. Moreover, with IPTG induction, the amount of plasmid recovered from transformants containing pUC4K_T7oriC further increased. These results indicate that the T7oriC cassette enhances the effect of increasing the copy number per host cell by inducing transcription from the reverse T7 promoter. Additionally, in FIG. 4(B), transformants containing pUC4K_PoriC_terG16 showed an improvement in the copy number per host cell compared to those containing pUC4K_T7oriC.

### Example 3

The effect of the T7oriC cassette on the copy number of pUC plasmid was examined by incorporating the ToriC cassette into plasmids with moderate copy numbers per host cell. Plasmids pETcoco-2 (Merck) and pET-dnaG (Non-Patent Literature 12) were used as plasmids with moderate copy numbers per host cell.

pETcoco-2 replicates from the F plasmid origin in the presence of glucose, resulting in 1 to 2 copies per cell, while it replicates from the RK-2 plasmid origin in the presence of arabinose, resulting in 20 to 50 copies per cell. On the other hand, pET-dnaG is a ColEl-type plasmid but has a lower copy number than the pUC plasmid which is modified to have high copy numbers.

Using the OriCiro Cell-Free Switching System, the T7oriC cassette was incorporated into pETcoco-2 through a homologous recombination reaction. The target region for the homologous recombination reaction in pETcoco-2 was a 60 bp region adjacent to the origin in pETcoco-2. First, using the T7oriC cassette as a template, PCR was performed with the primer sets listed in Table 4 to obtain DNA fragments with overlapping sequences homologous to the target region (60 bp) at both ends as amplification products. In the base sequences of each primer in Table 4, the regions indicated by uppercase letters represent the overlapping sequences.

TABLE 4

| | | SEQ |
|---|---|---|
| | | ID |
| Primer | Base sequence | NO: |
| pETcoco_ | ACACCGAGGTTACTCCGTTCTACAGGT | 29 |
| Fw | TACGACGACATGCTCTTCAGTAAACAC | |
| | CTCAGCgagctgttgacagagggtc | |
| pETcoco_ | TTGTCGATCAGACTATCAGCGTGAGAC | 30 |
| Rv | TACGATTCCATGCTGAGGTGTTTACTG | |
| | AAGAGCtaaaaaaacagcctccgaaag | |
| | gag | |

In the same manner as in Example 1, the T7oriC cassette fragment with added overlapping sequences was inserted into pETcoco-2 through a homologous recombination reaction to prepare the plasmid pETcoco-T7oriC.

Similarly, using the ColE1_Fw primer and ColE1_Rv primer, the PoriC_terG16 cassette with added overlapping sequences was inserted into pET-dnaG through a homologous recombination reaction to prepare the plasmid pET-dnaG-PoriC_terG16.

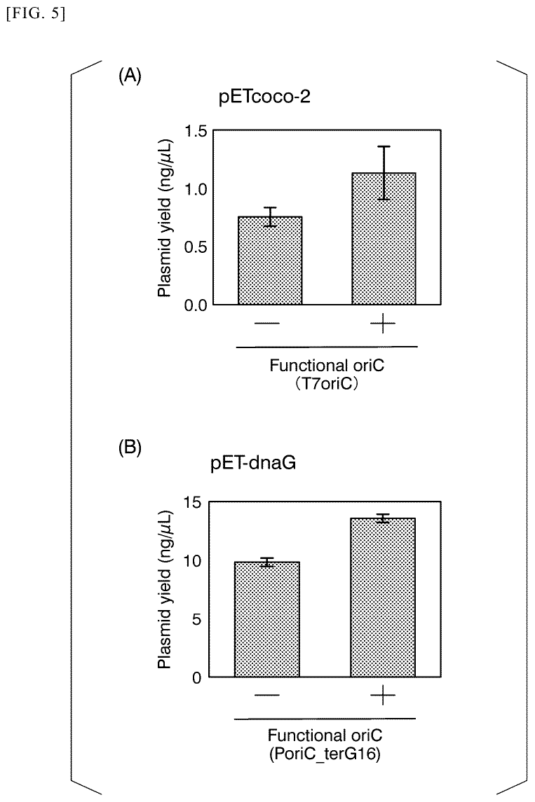

Transformants of Escherichia coli DH5α were obtained by introducing pETcoco-2 or pETcoco-T7oriC into the cells. The transformants were cultured in 10 mL of LB liquid medium containing 0.1% arabinose and 75 μg/mL ampicillin at 37° C. for 16 hours with shaking. The cultures were then processed in the same manner as in Example 1, and plasmid extraction was performed while equalizing the number of cells, followed by measurement of the plasmid concentration. The measurement results are shown in FIG. 5(A). Experiments were conducted using two independent colonies for each sample, and the average values and standard errors are shown.

Transformants of Escherichia coli DH5α were obtained by introducing pET-dnaG or pET-dnaG-PoriC_terG16 into the cells. The transformants were cultured in 4 mL of LB liquid medium containing 75 μg/mL ampicillin at 37° C. for 16 hours with shaking. The cultures were then processed in the same manner as in Example 1, and plasmid extraction was performed while equalizing the number of cells, followed by measurement of the plasmid concentration. The measurement results are shown in FIG. 5(B).

As shown in FIG. 5, the incorporation of the T7oriC cassette or the PoriC_terG16 cassette into any of the plasmids tended to increase the intracellular copy number.

### Example 4

A PoriC_sg cassette was prepared by inserting the gyrase-binding sequence (Mu-SGS) from bacteriophage Mu into the PoriC_terG16 cassette, and the effect of the PoriC_sg cassette on the copy number of pUC plasmid was examined.

A DNA fragment was prepared as a PoriC_sg cassette (582 bp, SEQ ID NO: 31), where the SGS was connected to the 5′ side of oriC in the PoriC_terG16 cassette sequence. The base sequence of the PoriC_sg cassette is shown in Table 5. In the table, the region indicated by uppercase letters at the 5′ end represents the Tac promoter sequence, the region indicated by uppercase letters at the 3′ end represents the fdhF terminator sequence, the underlined uppercase letters represent the SGS (with bold letters indicating the consensus sequence of SGS), and the region indicated by uppercase letters near the center represents the oriC sequence. The regions enclosed in squares represent the terG16 sequence and its complementary sequence.

TABLE 5

| | | SEQ |
|---|---|---|
| | | ID |
| cassette | Base sequence | NO: |
| PoriC_sg | GAGCTGTTGACAGAGGGTCATTTTCACACTATAATGcagtgaatcccaaaca | 31 |
| | 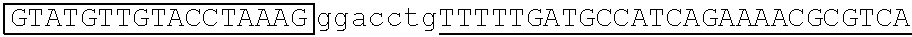 | |
| | GCGCCGCTCTGAGGCAATAAACAGAATCAGGCATAAAATCAGCCGCACAGATT | |
| | TTTTAAAACGCGCCACGGGATTTTTAAACCGGTATTTAACGGTGTATGAATCCC | |
| | GTTTTATCTTCCTTTggatcctggGTATTAAAAAGAAGATCTATTTATTTAGA | |
| | GATCTGTTCTATTGTGATCTCTTATTAGGATCGCACTGCCCTGTGGATAACAAG | |
| | GATCCGGCTTTTAAGATCAACAACCTGGAAAGGATCATTAACTGTGAATGATCG | |
| | GTGATCCTGGACCGTATAAGCTGGGATCAGAATGAGGGGTTATACACAACTCAA | |
| | AAACTGAACAACAGTTGTTCTTTGGATAACTACCGGTTGATCCAAGCTTCCTGA | |
| | 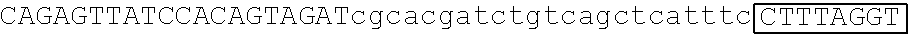 | |
| | 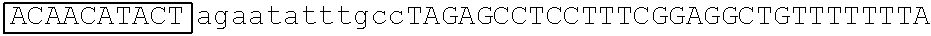 | |

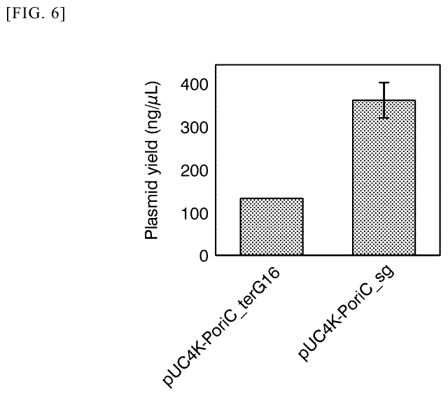

Using the homologous recombination reaction in the same manner as in Example 1, a DNA fragment containing the PoriC_sg cassette was prepared and incorporated into pUC4K to prepare the plasmid pUC4K_PoriC_sg. Transformants of Escherichia coli DH5α were obtained by introducing pUC4K-PoriC_terG16 or pUC4K_PoriC_sg into the cells. The transformants were cultured in 3 mL of LB liquid medium containing 75 μg/mL ampicillin at 37° C. for 16 hours with shaking. The cultures were then processed in the same manner as in Example 1, and plasmid extraction was performed while equalizing the number of cells, followed by measurement of the plasmid concentration. The measurement results are shown in FIG. 6.

In the case of pUC4K_PoriC_sg, experiments were conducted using five independent colonies, and the average values and standard errors are shown. For pUC4K-PoriC_terG16, a single colony was used. There was no significant difference in the turbidity of the cultures between the strain containing pUC4K-PoriC_terG16 and the strain containing pUC4K_PoriC_sg. As shown in FIG. 6, the incorporation of SGS increased the copy number of pUC4K-PoriC_terG16 by 2.7 times, demonstrating that the incorporation of SGS further increased the intracellular copy number. In Example 1, the copy number of pUC4K-PoriC_terG16 was estimated to be 1000 to 1800, so the copy number of pUC4K_PoriC_sg was estimated to have increased to about 2700 to 5000.

### Example 5

Using the oriC_ter cassette (SEQ ID NO: 19) shown in Table 1 of Example 1 as a template, PCR was performed with the primer sets listed in Table 6 to obtain a DNA fragment (oriCb) with 60 bp overlapping sequences homologous to the downstream of the ampicillin resistance gene promoter of pUC4K and no ter sequences. The regions indicated by uppercase letters in the base sequences of each primer in Table 6 represent the overlapping sequences. Using a homologous recombination reaction, the DNA fragment oriCb was incorporated into pUC4K to prepare the plasmid pUC4K_oriCb.

TABLE 6

| | | SEQ |
|---|---|---|
| | | ID |
| Primer | Base sequence | NO: |
| oriCb_F | TACATTCAAATATGTATCCGCTCATGA | 33 |
| | GACAATAACCCTGATAAATGCTTCAAT | |
| | AATATTtattaaaaagaagatctattt | |
| | atttagagatctgttc | |
| oriCb_R | CCGCAAAAAAGGGAATAAGGGCGACAC | 34 |
| | GGAAATGTTGAATACTCATACTCTTCC | |
| | TTTTTCatctactgtggataactctgt | |
| | cag | |

Similarly, using the primer sets listed in Table 7 instead of the primer sets listed in Table 6, a DNA fragment (oriCc) was prepared, which is an inverted form of oriCb without ter sequences. Using a homologous recombination reaction, the obtained DNA fragment oriCc was incorporated into pUC4K to prepare the plasmid pUC4K_oriCc. The regions indicated by uppercase letters in the base sequences of each primer in Table 7 represent the overlapping sequences.

TABLE 7

| | | SEQ |
|---|---|---|
| | | ID |
| Primer | Base sequence | NO: |
| oriCc_ | TACATTCAAATATGTATCCGCTCATGA | 35 |
| F | GACAATAACCCTGATAAATGCTTGAAT | |
| | AATATTatctactgtggataactctgt | |
| | cag | |
| oriCc_ | CCGCAAAAAAGGGAATAAGGGCGACAC | 36 |
| R | GGAAATGTTGÅATACTCATACTCTTCC | |
| | TTTTTCtattaaaaagaagatctattt | |
| | atttagagatctgttc | |

For the homologous recombination reaction, the oriC cassette fragment with added overlapping sequences was inserted into pUC4K using RecA family recombinase protein and 3′→5′ exonuclease in the same manner as the OriCiro Cell-Free Switching System. Wild-type Escherichia coli RecA (Patent Literature 8) was used as the RecA family recombinase protein, and exonuclease III was used as the 3′→5′ exonuclease.

For the homologous recombination reaction, 200 pM of pUC4K and 200 pM of the oriC cassette fragment with added overlapping sequences were added to a 5 μL reaction solution (containing 1 μM RecA, 80 mU/uL exonuclease III, 20 mM Tris-HCl (pH 8.0), 4 mM DTT, 1 mM magnesium acetate, 100 μM ATP, 4 mM creatine phosphate, 20 ng/μL creatine kinase, 50 mM potassium glutamate, 150 mM TMAC, 5% PEG8000, and 10 v/v % DMSO) and incubated at 37° C. for 30 minutes. Next, 0.5 μL of the reaction solution was mixed with 4.5 μL of a mixture containing 60 nM Tus and the components shown in Table 8 to prepare a 5 μL RCR amplification reaction solution. The RCR amplification reaction was carried out by incubating this solution at 30° C. for 16 hours. Tus was prepared and purified from Escherichia coli expression strains through affinity column chromatography and gel filtration column chromatography.

TABLE 8
Reaction mixture
Reaction buffer

| Tris-HCl (pH 8.0) | 20 | mM |
|---|---|---|
| Dithiothreitol | 8 | mM |
| Potassium glutamate | 150 | mM |
| Mg(OAc)₂ | 10 | mM |
| Creatine phosphate | 4 | mM |
| ATP | 1 | mM |
| GTP, CTP, UTP | each 1 mM | |
| dNTPs | each 0.1 mM | |
| tRNA | 50 | ng/μL |
| NAD | 0.25 | mM |
| Ammonium sulfate | 10 | mM |
| Bovine serum albumin (BSA) | 0.5 | mg/mL |
| Creatine kinase | 20 | ng/μL |

Enzymes

| | | |
|---|---|---|
| SSB | 400 | nM |
| IHF | 20 | nM |
| DnaG | 400 | nM |
| DnaN | 40 | nM |
| PolIII* | 5 | nM |
| DnaB, DnaC | 20 | nM |
| DnaA | 100 | nM |
| RNaseH | 10 | nM |
| Ligase | 50 | nM |
| PolI | 50 | nM |
| GyrA, GyrB | 50 | nM |
| Topo IV | 5 | nM |
| Topo III | 50 | nM |
| RecQ | 50 | nM |

In Table 8, SSB represents SSB from Escherichia coli, IHF represents a complex of IhfA and IhfB from Escherichia coli, DnaG represents DnaG from Escherichia coli, DnaN represents DnaN from Escherichia coli, Pol III* represents the DNA polymerase III* complex from Escherichia coli consisting of DnaX, HolA, HolB, HolC, HolD, DnaE, DnaQ, and HolE, DnaB represents DnaB from Escherichia coli, DnaC represents DnaC from Escherichia coli, DnaA represents DnaA from Escherichia coli, RNaseH represents RNaseH from Escherichia coli, Ligase represents DNA ligase from Escherichia coli, Pol I represents DNA polymerase I from Escherichia coli, GyrA represents GyrA from Escherichia coli, GyrB represents GyrB from Escherichia coli, Topo IV represents the complex of ParC and ParE from Escherichia coli, Topo III represents topoisomerase III from Escherichia coli, and RecQ represents RecQ from Escherichia coli.

SSB was prepared and purified from Escherichia coli expression strains through ammonium sulfate precipitation and ion exchange column chromatography.

IHF was prepared and purified from co-expression strains of IhfA and IhfB from Escherichia coli through ammonium sulfate precipitation and affinity column chromatography.

DnaG was prepared and purified from Escherichia coli expression strains through ammonium sulfate precipitation, anion exchange column chromatography, and gel filtration column chromatography.

DnaN was prepared and purified from Escherichia coli expression strains through ammonium sulfate precipitation and anion exchange column chromatography.

Pol III* was prepared and purified from Escherichia coli co-expression strains of DnaX, HolA, HolB, HolC, HolD, DnaE, DnaQ, and HolE through ammonium sulfate precipitation, affinity column chromatography, and gel filtration column chromatography.

DnaB and DnaC were prepared and purified from Escherichia coli co-expression strains of DnaB and DnaC through ammonium sulfate precipitation, affinity column chromatography, and gel filtration column chromatography.

DnaA was prepared and purified from Escherichia coli expression strains through ammonium sulfate precipitation, dialysis precipitation, and gel filtration column chromatography.

GyrA and GyrB were prepared and purified from a mixture of Escherichia coli expression strains of GyrA and GyrB through ammonium sulfate precipitation, affinity column chromatography, and gel filtration column chromatography.

Topo IV was prepared and purified from a mixture of Escherichia coli expression strains of ParC and ParE through ammonium sulfate precipitation, affinity column chromatography, and gel filtration column chromatography.

Topo III was prepared and purified from Escherichia coli expression strains through ammonium sulfate precipitation and affinity column chromatography.

RecQ was prepared and purified from Escherichia coli expression strains through ammonium sulfate precipitation, affinity column chromatography, and gel filtration column chromatography.

RNaseH, Ligase, and Pol I were commercial enzymes derived from Escherichia coli (Takara Bio).

After completing the RCR amplification reaction, a portion of the RCR amplification reaction solution (0.4 μL) was diluted tenfold with RCR reaction buffer (the “reaction buffer” composition shown in Table 8) and incubated at 30° C. for 30 minutes.

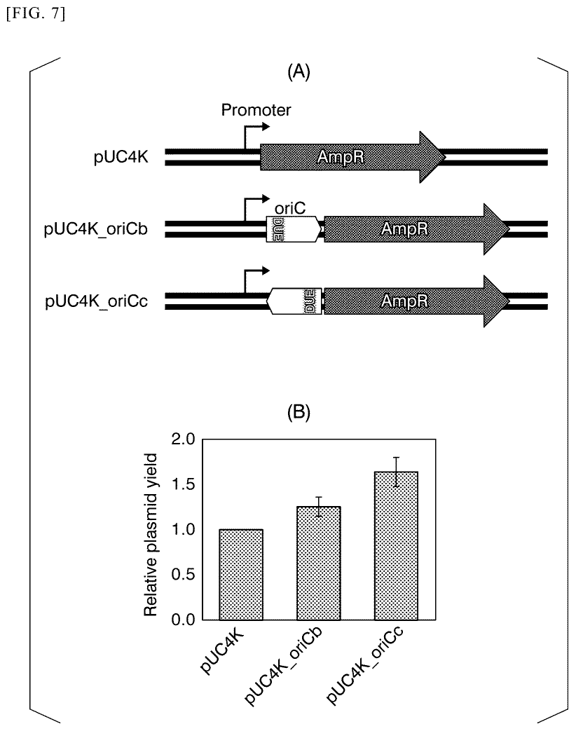

A schematic diagram of the structures of pUC4K, pUC4K_oriCb, and pUC4K_oriCc is shown in FIG. 7(A). In FIG. 7(A), “DUE” indicates the double-strand unwinding element region of oriC. Plasmids pUC4K_oriCb and pUC4K_oriCc, as shown in FIG. 7(A), both have oriC inserted downstream of the ampicillin resistance gene promoter inherent in pUC4K, with the only difference being the orientation of oriC. These plasmids were introduced into Escherichia coli DH5a cells using the same method as in Example 1, except that LB agar plates containing 50 μg/mL kanamycin instead of 100 μg/mL carbenicillin were used. In each case, colonies of a size comparable to those formed with the introduction of pUC4K were observed, and no growth inhibition of Escherichia coli was observed.

The obtained colonies were cultured in LB liquid medium using the same method as in Example 1, except that the medium contained 50 μg/mL kanamycin instead of 50 μg/mL carbenicillin. There was no significant difference in the turbidity of the cultures among all the strains used. Plasmid extraction was performed from the cultures after culturing while equalizing the number of the cells using the same method as in Example 1, and the concentration of the obtained plasmid DNA solution was measured. The measurement results are shown in FIG. 7(B). Plasmid extraction was performed from four independent colonies, and the relative plasmid concentration compared to pUC4K was determined, with the average values and standard errors shown.

As shown in FIG. 7(B), the copy numbers of pUC4K_oriCb and pUC4K_oriCc per Escherichia coli cell were higher than that of pUC4K. These results demonstrate that the intracellular copy number of the plasmid can be increased by combining: any general gene promoter, such as the promoter of the ampicillin resistance gene inherent in the plasmid (a constitutive promoter derived from Escherichia coli); and oriC, such that transcription from the promoter flows into oriC. Furthermore, it was shown that the intracellular copy number of the plasmid can be increased regardless of the orientation of the promoter and oriC, as long as transcription from the promoter flows into oriC.

1. A DNA cassette comprising: a replication origin sequence capable of binding to an enzyme with DnaA activity; a first promoter sequence; and optionally a gyrase-binding sequence, wherein:
transcription from the first promoter sequence flows into the replication origin sequence, and
when the DNA cassette lacks the gyrase-binding sequence, the distance between the 3′ terminal base of the first promoter sequence and the terminal base of the replication origin sequence is within 450 bases; and
when the DNA cassette contains the gyrase-binding sequence, the distance between the 3′ terminal base of the first promoter sequence and the terminal base of the replication origin sequence is within 2000 bases.

2. The DNA cassette according to claim 1, wherein the DNA cassette contains the gyrase-binding sequence.

3. The DNA cassette according to claim 2, wherein the gyrase-binding sequence is derived from bacteriophage Mu.

4. The DNA cassette according to claim 1, further comprising a complementary sequence of a second promoter sequence on the 3′ side of the first promoter sequence.

5. A plasmid comprising the DNA cassette according to claim 1 and a plasmid replication origin.

6. (canceled)

7. The plasmid according to claim 5, wherein the DNA cassette contains the gyrase-binding sequence.

8. The plasmid according to claim 5, wherein the plasmid replication origin is of the ColE1 type.

9. The plasmid according to claim 5, wherein the distance between the first promoter sequence and the replication origin sequence is within 300 bases.

10. A bacterium comprising the plasmid according to claim 5.

11. The bacterium according to claim 10, wherein the bacterium is Escherichia coli.

12. A method for producing a plasmid, comprising culturing the bacterium according to claim 10 and recovering the plasmid from the resulting culture.

13. A method for producing single-stranded RNA, comprising producing a plasmid by the method according to claim 12 and obtaining RNA by transcription from the plasmid.

14. A method for preparing the plasmid according to claim 5, the method comprising:
introducing the DNA cassette into a plasmid comprising a plasmid replication origin.

15. The method according to claim 14, wherein
the DNA cassette is introduced into the plasmid such that transcription from the promoter sequence of the plasmid flows into the replication origin sequence.

16. The method according to claim 15, wherein the DNA cassette comprises the gyrase-binding sequence.

17. (canceled)

18. The method for preparing a plasmid according to claim 15, wherein introducing the DNA cassette into the plasmid comprises:
providing a reaction solution comprising the plasmid, the DNA cassette, a protein with RecA family recombinase activity, and an exonuclease, and
incubating the reaction solution to perform homologous recombination, wherein:
the plasmid contains regions Ha and Hb, the region Hb being located downstream of the region Ha, and
the DNA cassette contains a homologous region corresponding to the region Ha and a homologous region corresponding to the region Hb, the latter being positioned downstream of the former.

19. A DNA cassette for preparing high-copy-number plasmid, comprising a replication origin sequence capable of binding to an enzyme with DnaA activity; and a gyrase-binding sequence, wherein:
the length of the cassette being 1000 base pairs or less.

20. A DNA cassette comprising: a replication origin sequence capable of binding to an enzyme with DnaA activity; a first promoter sequence; and further comprising
a terminator sequence, a pair of ter sequences, or both, wherein:
transcription from the first promoter sequence flows into the replication origin sequence,
the distance between the 3′ terminal base of the first promoter sequence and the terminal base of the replication origin sequence is within 200 bases, and
when present, the terminator sequence is located downstream of the first promoter sequence, and the distance between the 3′ terminal base of the first promoter sequence and the 5′ terminal base of the terminator sequence being within 600 bases.

21. The DNA cassette according to claim 20, wherein the pair of ter sequences are present and are each inserted outward with respect to the replication origin sequence.

22-23. (canceled)


SEQUENCE LISTING (36 sequences)

SEQ ID NO: 1 - DNA, 14 bp, synthetic construct
  source 1..14: mol_type = other DNA
  misc_difference 2: note = n is a, c, g, or t
  misc_difference 3: note = r is a or g
  misc_difference 4: note = w is a or t
  misc_difference 13: note = k is g or t
gnrwgttgtaacka

SEQ ID NO: 2 - DNA, 14 bp, synthetic construct
  source 1..14: mol_type = other DNA
  misc_difference 2: note = k is g or t
  misc_difference 4: note = w is a or t
  misc_difference 13: note = k is g or t
gkawgttgtaacka

SEQ ID NO: 3 - DNA, 14 bp, synthetic construct
  source 1..14: mol_type = other DNA
gtatgttgtaacta

SEQ ID NO: 4 - DNA, 18 bp, synthetic construct
  source 1..18: mol_type = other DNA
agtatgttgtaactaaag

SEQ ID NO: 5 - DNA, 14 bp, synthetic construct
  source 1..14: mol_type = other DNA
ggatgttgtaacta

SEQ ID NO: 6 - DNA, 14 bp, synthetic construct
  source 1..14: mol_type = other DNA
gtatgttgtaacga

SEQ ID NO: 7 - DNA, 14 bp, synthetic construct
  source 1..14: mol_type = other DNA
ggatgttgtaacta

SEQ ID NO: 8 - DNA, 14 bp, synthetic construct
  source 1..14: mol_type = other DNA
ggaagttgtaacga

SEQ ID NO: 9 - DNA, 14 bp, synthetic construct
  source 1..14: mol_type = other DNA
gtaagttgtaacga

SEQ ID NO: 10 - DNA, 14 bp, synthetic construct
  source 1..14: mol_type = other DNA
  misc_difference 2: note = n is a, c, g, or t
  misc_difference 3: note = r is a or g
  misc_difference 4: note = w is a or t
  misc_difference 13: note = k is g or t
gnrwgttgtaccka

SEQ ID NO: 11 - DNA, 14 bp, synthetic construct
  source 1..14: mol_type = other DNA
gtatgttgtaccta

SEQ ID NO: 12 - DNA, 23 bp, synthetic construct
  source 1..23: mol_type = other DNA
  misc_difference 3: note = w is a or t
  misc_difference 4: note = r is a or g
  misc_difference 6..10: note = n is a, c, g or t
  misc_difference 11: note = y is t or c
  misc_difference 12: note = n is a, c, g or t
  misc_difference 19: note = n is a, c, g or t
acwrannnnnynatgtacnaaat

SEQ ID NO: 13 - DNA, 23 bp, synthetic construct
  source 1..23: mol_type = other DNA
  misc_difference 8: note = r is a or g
  misc_difference 10: note = w is a or t
  misc_difference 12: note = y is c or t
actaattrawcyatgtactaaat

SEQ ID NO: 14 - DNA, 28 bp, synthetic construct
  source 1..28: mol_type = other DNA
  misc_difference 8: note = r is a or g
  misc_difference 10: note = w is a or t
  misc_difference 12: note = y is c or t
actaattrawcyatgtactaaattttca

SEQ ID NO: 15 - DNA, 30 bp, synthetic construct
  source 1..30: mol_type = other DNA
gaactaattaaactatgtactaaattttca

SEQ ID NO: 16 - DNA, 30 bp, synthetic construct
  source 1..30: mol_type = other DNA
atactaattgatccatgtactaaattttca

SEQ ID NO: 17 - DNA, 264 bp, Escherichia coli
  source 1..264: mol_type = genomic DNA
tattaaaaagaagatctatttatttagagatctgttctattgtgatctcttattaggatcgcactgccctgtggataacaaggatccggcttttaagatcaacaacctggaaaggatcattaactgtgaatgatcggtgatcctggaccgtataagctgggatcagaatgaggggttatacacaactcaaaaactgaacaacagttgttctttggataactaccggttgatccaagcttcctgacagagttatccacagtagat

SEQ ID NO: 18 - DNA, 18 bp, synthetic construct
  source 1..18: mol_type = other DNA
agtatgttgtaactaaag

SEQ ID NO: 19 - DNA, 378 bp, synthetic construct
  source 1..378: mol_type = other DNA
cgcgtcagcgggtgttggcgggtgtcggggctggcttaacagtatgttgtaactaaagtattaaaaagaagatctatttatttagagatctgttctattgtgatctcttattaggatcgcactgccctgtggataacaaggatccggcttttaagatcaacaacctggaaaggatcattaactgtgaatgatcggtgatcctggaccgtataagctgggatcagaatgaggggttatacacaactcaaaaactgaacaacagttgttctttggataactaccggttgatccaagcttcctgacagagttatccacagtagatctttagttacaacatactatgcggcatcagagcagattgtactgagagtgcaccat

SEQ ID NO: 20 - DNA, 265 bp, Escherichia coli
  source 1..265: mol_type = genomic DNA
gtattaaaaagaagatctatttatttagagatctgttctattgtgatctcttattaggatcgcactgccctgtggataacaaggatccggcttttaagatcaacaacctggaaaggatcattaactgtgaatgatcggtgatcctggaccgtataagctgggatcagaatgaggggttatacacaactcaaaaactgaacaacagttgttctttggataactaccggttgatccaagcttcctgacagagttatccacagtagat

SEQ ID NO: 21 - DNA, 18 bp, synthetic construct
  source 1..18: mol_type = other DNA
agtatgttgtacctaaag

SEQ ID NO: 22 - DNA, 36 bp, synthetic construct
  source 1..36: mol_type = other DNA
gagctgttgacagagggtcattttcacactataatg

SEQ ID NO: 23 - DNA, 30 bp, synthetic construct
  source 1..30: mol_type = other DNA
tacagcctcctttcggaggctgttttttta

SEQ ID NO: 24 - DNA, 426 bp, synthetic construct
  source 1..426: mol_type = other DNA
gagctgttgacagagggtcattttcacactataatgcagtgaatcccaaacagtatgttgtacctaaagggatcctgggtattaaaaagaagatctatttatttagagatctgttctattgtgatctcttattaggatcgcactgccctgtggataacaaggatccggcttttaagatcaacaacctggaaaggatcattaactgtgaatgatcggtgatcctggaccgtataagctgggatcagaatgaggggttatacacaactcaaaaactgaacaacagttgttctttggataactaccggttgatccaagcttcctgacagagttatccacagtagatcgcacgatctgtcagctcatttcctttaggtacaacatactagaatatttgcctacagcctcctttcggaggctgttttttta

SEQ ID NO: 25 - DNA, 390 bp, synthetic construct
  source 1..390: mol_type = other DNA
gagctgttgacagagggtcattttcacactataatgcagtgaatcccaaacggatcctgggtattaaaaagaagatctatttatttagagatctgttctattgtgatctcttattaggatcgcactgccctgtggataacaaggatccggcttttaagatcaacaacctggaaaggatcattaactgtgaatgatcggtgatcctggaccgtataagctgggatcagaatgaggggttatacacaactcaaaaactgaacaacagttgttctttggataactaccggttgatccaagcttcctgacagagttatccacagtagatcgcacgatctgtcagctcatttcagaatatttgcctacagcctcctttcggaggctgttttttta

SEQ ID NO: 26 - DNA, 79 bp, synthetic construct
  source 1..79: mol_type = other DNA
tgaccaaaatcccttaacgtgagttttcgttccactgagcgtcagaccccgtagaaaagagagctgttgacagagggtc

SEQ ID NO: 27 - DNA, 84 bp, synthetic construct
  source 1..84: mol_type = other DNA
tttttgtttgcaagcagcagattacgcgcagaaaaaaaggatctcaagaagatcctttgataaaaaaacagcctccgaaaggag

SEQ ID NO: 28 - DNA, 428 bp, synthetic construct
  source 1..428: mol_type = other DNA
gagctgttgacagagggtcattttcacactataatgccgctatagtgagtcgtagtatgttgtacctaaagggatcctgggtattaaaaagaagatctatttatttagagatctgttctattgtgatctcttattaggatcgcactgccctgtggataacaaggatccggcttttaagatcaacaacctggaaaggatcattaactgtgaatgatcggtgatcctggaccgtataagctgggatcagaatgaggggttatacacaactcaaaaactgaacaacagttgttctttggataactaccggttgatccaagcttcctgacagagttatccacagtagatcgcacgatctgtcagctcatttcctttaggtacaacatactagaatatttgcctacagcctcctttcggaggctgttttttta

SEQ ID NO: 29 - DNA, 79 bp, synthetic construct
  source 1..79: mol_type = other DNA
acaccgaggttactccgttctacaggttacgacgacatgctcttcagtaaacacctcagcgagctgttgacagagggtc

SEQ ID NO: 30 - DNA, 84 bp, synthetic construct
  source 1..84: mol_type = other DNA
ttgtcgatcagactatcagcgtgagactacgattccatgctgaggtgtttactgaagagctaaaaaaacagcctccgaaaggag

SEQ ID NO: 31 - DNA, 582 bp, synthetic construct
  source 1..582: mol_type = other DNA
gagctgttgacagagggtcattttcacactataatgcagtgaatcccaaacagtatgttgtacctaaagggacctgtttttgatgccatcagaaaacgcgtcagcgccgctctgaggcaataaacagaatcaggcataaaatcagccgcacagattttttaaaacgcgccacgggatttttaaaccggtatttaacggtgtatgaatcccgttttatcttcctttggatcctgggtattaaaaagaagatctatttatttagagatctgttctattgtgatctcttattaggatcgcactgccctgtggataacaaggatccggcttttaagatcaacaacctggaaaggatcattaactgtgaatgatcggtgatcctggaccgtataagctgggatcagaatgaggggttatacacaactcaaaaactgaacaacagttgttctttggataactaccggttgatccaagcttcctgacagagttatccacagtagatcgcacgatctgtcagctcatttcctttaggtacaacatactagaatatttgcctacagcctcctttcggaggctgttttttta

SEQ ID NO: 32 - DNA, 20 bp, synthetic construct
  source 1..20: mol_type = other DNA
  misc_difference 1: note = r is a or g
  misc_difference 2..4: note = n is a, c, g, or t
  misc_difference 5: note = r is a or g
  misc_difference 6: note = n is a, c, g, or t
  misc_difference 7: note = r is a or g
  misc_difference 8: note = k is g or t
  misc_difference 10: note = r is a or g
  misc_difference 11: note = y is c or t
  misc_difference 13: note = k is g or t
  misc_difference 14: note = y is c or t
  misc_difference 15: note = n is a, c, g, or t
  misc_difference 16: note = y is c or t
  misc_difference 17: note = n is a, c, g, or t
  misc_difference 18: note = k is g or t
  misc_difference 19: note = n is a, c, g, or t
  misc_difference 20: note = y is c or t
rnnnrnrkgryckynynkny

SEQ ID NO: 33 - DNA, 97 bp, synthetic construct
  source 1..97: mol_type = other DNA
tacattcaaatatgtatccgctcatgagacaataaccctgataaatgcttcaataatatttattaaaaagaagatctatttatttagagatctgttc

SEQ ID NO: 34 - DNA, 84 bp, synthetic construct
  source 1..84: mol_type = other DNA
ccgcaaaaaagggaataagggcgacacggaaatgttgaatactcatactcttcctttttcatctactgtggataactctgtcag

SEQ ID NO: 35 - DNA, 84 bp, synthetic construct
  source 1..84: mol_type = other DNA
tacattcaaatatgtatccgctcatgagacaataaccctgataaatgcttcaataatattatctactgtggataactctgtcag

SEQ ID NO: 36 - DNA, 97 bp, synthetic construct
  source 1..97: mol_type = other DNA
ccgcaaaaaagggaataagggcgacacggaaatgttgaatactcatactcttcctttttctattaaaaagaagatctatttatttagagatctgttc